(19) United States
(12) Patent Application Publication
(10) Pub. No.: US 2013/0336957 A1
(43) Pub. Date: Dec. 19, 2013

(54) NOVEL PURIFICATION OF HUMAN, HUMANIZED, OR CHIMERIC ANTIBODIES USING PROTEIN A AFFINITY CHROMATOGRAPHY

(71) Applicant: AbbVie, Inc., North Chicago, IL (US)

(72) Inventors: Chen Wang, Shrewsbury, MA (US); Susan Lacy, North Chicago, IL (US); Randolf Huelsman, North Chicago, IL (US)

(73) Assignee: AbbVie, Inc., North Chicago, IL (US)

(21) Appl. No.: 13/898,929

(22) Filed: May 21, 2013

Related U.S. Application Data
(60) Provisional application No. 61/649,687, filed on May 21, 2012.
(60) Provisional application No. 61/768,714, filed on Feb. 25, 2013.

(51) Int. Cl. C07K 16/00

(52) U.S. Cl. CPC C07K 16/00
USPC 424/130.1; 530/387.1; 530/387.3

(57) ABSTRACT

Disclosed herein are compositions and methods for the isolation and purification of antibodies from a sample matrix. In particular, the present invention relates to compositions and methods for isolating and purifying antibodies exhibiting low or high binding capacity for Protein A resin. In certain embodiments, the methods herein employ a kosmotropic salt solution, an affinity chromatographic step, and may include one or more additional chromatography and/or filtration steps to achieve the desired degree of purification. The present invention is also directed toward pharmaceutical compositions comprising one or more antibodies purified by a method described herein.

### CROSS REFERENCE TO RELATED APPLICATIONS

The present application claims priority to U.S. Provisional Application No. 61/649,687, filed on May 21, 2012, and U.S. Provisional Application No. 61/768,714, filed Feb. 25, 2013, the disclosures of both of which are incorporated by reference in their entirety.

### 1. BACKGROUND OF THE INVENTION

Protein A chromatographic resins are often used in commercial purification processes for pharmaceutical grade monoclonal antibodies. Protein A is a bacterial cell wall protein that binds to mammalian antibodies, primarily through hydrophobic interactions along with hydrogen bonding and two salt bridges with the antibodies' Fc regions. Thus, in the context of chromatographic purification, Protein A resins allow for the affinity-based retention of antibodies on a chromatographic support, while the majority of the components in a clarified harvest flow past the support and can be discarded. The retained antibodies can then be eluted from the chromatographic support by disrupting the antibody-Protein A interaction and subjected to further purification steps, e.g., those relying on charge (ion exchange chromatography), hydrophobic characteristics (hydrophobic interaction chromatography), and/or size (ultrafiltration).

Protein A-based affinity purification finds particular use in connection with a variety of commercially relevant immunoglobulin isotypes, particularly IgG1, IgG2, and IgG4. However, not all antibodies, including not all IgG1, IgG2, and IgG4 isotype immunoglobulins, are capable of binding Protein A with equal affinity. For instance, mouse IgG1, canine, horse or cow IgG does not bind as strongly as a typical human IgG1 to Protein A. Consequently, those antibodies exhibiting weak binding strength for Protein A resin can result in low binding capacity under standard Protein A operating conditions, and thus demand substantially larger Protein A column to process a given batch of antibody feed. Since Protein A capture is one of the most expensive steps in antibody downstream processing, using excess amount of Protein A resin will significantly increase its operating cost and create inefficiencies in conventional Protein A-based purification strategies. Hence, there is a present need for high-efficiency methods of purifying antibodies exhibiting weak binding strength and low binding capacity for Protein A resin. The present invention addresses this need.

### 2. SUMMARY OF THE INVENTION

The present invention is directed to compositions and methods for purifying antibodies from a sample matrix. In certain embodiments, the methods are disclosed that are directed to improving Protein A performance for the purification of antibodies. In particular, the present invention relates to compositions and methods for purifying antibodies exhibiting weak binding strength and low binding capacity for Protein A resin. In certain embodiments, the present invention is directed to enhancing the amount of an antibody of interest retained on a Protein A resin where such weak binding strength for Protein A ligand results in about 2-10 fold lower binding capacity than typical human IgGs (except for human IgG3) on such resins under standard operating conditions. In certain embodiments, the present invention is also directed to enhance the binding capacity for antibodies that have high affinity for Protein A ligand under typical Protein A capture operating conditions, including but not limited to chimeric, humanized or human antibodies, as well as to enhance the separation of such antibodies from impurities, such as host cell proteins (HCPs).

In certain embodiments, a kosmotropic salt, which contributes to the stability and structure of water-water interactions and causes water molecules to favorably interact with macromolecules such as proteins and also stabilizes the intermolecular interactions, is employed to enhance the hydrophobic interaction between the antibody and Protein A, and thereby increasing the retention of the antibody of interest on the Protein A resin. In certain embodiments, the concentration of the antibody of interest in the sample exposed to a Protein A resin is increased to enhance the retention of the antibody of interest on the Protein A resin. The increase of antibody concentration can be achieved via a membrane ultrafiltration step. In certain embodiments, a combination of a kosmotropic salt solution and of an increased concentration of the antibody of interest in the sample is employed to enhance the retention of the antibody of interest on the Protein A resin. In certain embodiments, a kosmotropic salt is employed to enhance the reduction of HCPs in the Protein A eluate for antibodies with high affinity for Protein A resin.

In certain embodiments, the purification strategies of the present invention may include one or more additional chromatography and/or filtration steps to achieve a desired degree of purification. For example, in certain embodiments, the chromatography step(s) can include one or more step of ion exchange chromatography and/or hydrophobic interaction chromatography. In addition, in certain embodiments, the present invention is directed toward pharmaceutical compositions comprising one or more antibodies purified by methods described herein.

In certain embodiments, the present invention is directed toward methods of purifying an antibody from a sample matrix such that the resulting antibody composition is substantially free of host cell proteins (“HCPs”). In certain embodiments, the sample matrix (or simply “sample”) comprises a cell line harvest wherein the cell line is employed to produce specific antibodies of the present invention. In certain embodiments, the sample matrix is prepared from a cell line used to produce an antibody that has a weak binding strength and low binding capacity for Protein A resin. In certain embodiments, the antibody of interest is a canine antibody, a feline antibody, a horse antibody, a cow antibody, or a multivalent antibody. In certain embodiments, the sample matrix is prepared from a cell line used to produce an antibody that has high binding affinity for Protein A resin. In certain embodiments, the antibody of interest is a chimeric antibody, a humanized antibody, a human antibody. In certain embodiments, the antibody can be a multivalent antibody, a dual-variable domain antibody, or an antigen-binding portion thereof.

In certain embodiments, the present invention involves clarifying a harvest sample containing immunoglobulin antibody of interest through centrifugation and/or depth filtration, concentrating the clarified harvest via ultrafiltration, and then mixing it with a kosmotropic salt solution to form a conditioned clarified (or primary recovery) sample. The conditioned primary recovery sample is then contacted with a Protein A resin and the resin is washed to remove the components of the sample that are not retained on the resin. The antibody of interest can then be eluted from the resin by disrupting the antibody-Protein A interaction. In certain of such embodiments, the kosmotropic salt solution comprises at least one kosmotropic salt. Examples of suitable kosmotropic salts include, but are not limited to, ammonium sulfate ((NH₄)₂SO₄), sodium sulfate (Na₂SO₄), sodium citrate (NaCitrate), potassium sulfate (K₂SO₄), potassium phosphate (K₃PO₄), sodium phosphate (Na₃PO₄), or a combination thereof. In certain embodiments, the kosmotropic salt is ammonium sulfate; in certain embodiments, the kosmotropic salt is sodium sulfate; and in certain embodiments, the kosmotropic salt is sodium citrate.

In certain embodiments, the kosmotropic salt(s) is present in the kosmotropic salt solution at a concentration of from about 0.3 M to about 1.1 M. In certain embodiments, the kosmotropic salt(s) is present in the kosmotropic salt solution at a concentration of about 0.5 M. In certain embodiments, the kosmotropic salt(s) is present in the kosmotropic salt solution at a concentration of about 0.8 M. In certain embodiments, the kosmotropic salt(s) is present in the kosmotropic salt solution at a concentration of about 1 M.

In certain embodiments, the present invention employs a step of preconditioning the harvest sample containing antibody of interest such that the concentration of the antibody is increased and then loading this sample to the Protein A chromatography resin, given that, as disclosed herein, antibodies exhibiting low binding capacity for Protein A resin also exhibit concentration dependent Protein A retention. The Protein A resin exposed to a sample in this manner can then be washed to remove the components of the sample that are not bound to the resin. The antibody of interest can then be eluted from the resin by disrupting the antibody-Protein A interaction. In certain embodiments, the concentration of the antibody of interest in the sample that is contacted to an affinity chromatography resin is increased as compared to conventional purification strategies, such as, but not limited to, concentrations of from about 1 to about 8 g/L, about 1.5 g/L to about 5.8 g/L, about 1.7 g/L to about 5.8 g/L, about 1.9 g/L to about 5.45 g/L, about 1.9 g/L to about 4.95 g/L, about 1.9 g/L to about 4.7 g/L, about 1.9 g/L to about 4.5 g/L, or about 1.9 g/L to about 3.6 g/L. In certain embodiments, the concentration is about 1.5 g/L, about 1.7 g/L, about 1.9 g/L, about 3.6 g/L, about 4.5 g/L, about 4.7 g/L, about 4.95 g/L, about 5.3 g/L, about 5.45 g/L, about 5.5 g/L, or about 5.8 g/.

In certain embodiments, the present invention involves subjecting a pre-concentrated sample matrix comprising the antibody of interest to a kosmotropic salt solution, thus forming a conditioned primary recovery sample, and subsequently loading such sample comprising an increased concentration of the antibody of interest and kosmotropic salt to a Protein A resin.

In certain embodiments, a filtration step using depth filters containing cationic charge functionality will follow the Protein A affinity chromatography step to remove any turbidity and impurities including HCPs, DNA, aggregates and leached Protein A. Examples of such depth filters include but are not limited to Millistak+X0HC, F0HC, A1HC, B1HC filters from EMD Millipore, and VR05, VR07, Zeta Plus 30ZA/60ZA and 60ZA/90ZA from 3M. In certain embodiments, the depth filter is Millistak+ X0HC Pod filter.

In certain embodiments, a hydrophobic interaction chromatography (“HIC”) step follows Protein A affinity chromatography instead of the depth filtration. Such HIC step may employ a resin or a membrane coupled with defined hydrophobic ligands. In certain embodiments, the HIC step comprises the use of a column of packed resin. Example of such a resin include, but are not limited to, Phenyl Sepharose (such as Phenyl Sepharose™ 6 Fast Flow, Phenyl Sepharose™ High Performance), Octyl Sepharose™ High Performance, Fractogel™ EMD Propyl, Fractogel™ EMD Phenyl, Macro-Prep™ Methyl, Macro-Prep™ t-Butyl Supports, WP HI-Propyl (C₃)™, and Toyopearl™ Ether, Phenyl or Butyl. In certain embodiments, the column is Phenyl Sepharose HP or Capto Phenyl. HIC resin also comprises at least one hydrophobic group. Examples of suitable include, but are not limited to alkyl-, aryl-, aromatic-groups, and a combination thereof. It is possible that the antibodies of interest have formed aggregates during the isolation/purification process. Such hydrophobic interaction chromatographic steps can effectively remove aggregates and other process-related impurities. In certain embodiments, the procedures of the instant invention employ a high salt buffer which promotes interaction of the antibodies (or aggregates thereof) with the HIC resin. In certain embodiments, the column can be eluted using lower concentrations of salt. In certain embodiments, the column can be operated in flow-through mode at which the salt condition of the load sample is carefully selected such that the aggregates, HCPs and other impurities are retained to the column while the product flows through the column.

In certain embodiments, an ion exchange chromatography step will follow the post-Protein A capture depth filtration or the post-Protein A hydrophobic interaction chromatography step, thereby forming an ion exchange eluate sample. In certain embodiments, the ion exchange step is either a cation exchange step or an anion exchange step. In certain embodiments, the ion exchange step is a single ion exchange chromatographic step or can include multiple ion exchange steps such as a cation exchange step followed by an anion exchange step or vise versa. In one aspect, the ion exchange step is a one step procedure. In certain embodiments, the ion exchange step involves a two step ion exchange process. A suitable cation exchanger is a resin or membrane whose stationary phase comprises anionic groups. Examples of such a cation exchange functional group include, but are not limited to carboxymethyl (CM), sulfoethyl (SE), sulfopropyl (SP), phosphate (P) and sulfonate (S). A suitable anion exchanger is a resin or membrane whose stationary phase comprises cationic groups. Examples of such an anion exchange functional group include, but are not limited to, diethylaminoethyl (DEAE), quaternary aminoethyl (QAE), and quaternary amine (Q) groups. In certain embodiments, the anion exchange resin is Capto Q or Q Sepharose Fast Flow™. One or more ion exchange step further isolates antibodies by reducing impurities such as host cell proteins, aggregates, DNA, and where applicable, affinity matrix protein (e.g. Protein A).

The ion exchange eluate sample is further subject to viral filtration. Filters well known to those skilled in the art can be used in this embodiment. Examples of viral filters include, but are not limited to, Virosart CPV filter from Sartorius, Virosolve from Millipore, Ultipor DV20 or DV50 from Pall, Planova 20N and 50N or BioEx from Asahi. The viral filtrate is then subjected to ultrafiltration and diafiltration for final formulation of the drug product. Membrane devices well known to those skilled in the art can be used in this embodiment.

The purity of the antibodies of interest in the resultant sample product can be analyzed using methods well known to those skilled in the art, e.g., size-exclusion chromatography, Paros™ A or Poros G HPLC Assay, HCP ELISA, Protein A ELISA, and western blot analysis.

In certain embodiments, the invention is directed to one or more pharmaceutical compositions comprising an isolated antibody and an acceptable carrier. In certain embodiments, the compositions further comprise one or more pharmaceutical agents.

### 3. BRIEF DESCRIPTIONS OF THE DRAWINGS

FIG. 1 depicts a two-column purification process for the present invention.

FIG. 2 depicts a three-column purification process for the present invention.

FIG. 3 depicts the effects of the load protein concentration on the static binding capacity of a weak Protein A binding monoclonal antibody to MabSelect SuRe Protein A resin.

FIG. 4 depicts the effect of various kosmotropic salts and their concentrations on static binding capacity of a weak Protein A binding monoclonal antibody to MabSelect SuRe Protein A resin.

FIG. 5 depicts the effects of (NH₄)₂SO₄, protein concentration and flow rates on dynamic binding capacity of a weak Protein A binding monoclonal antibody on MabSelect SuRe Protein A column.

FIG. 6 depicts the effect of various kosmotropic salt solution comprising ammonium sulfate, sodium sulfate, or sodium citrate on the binding capacity of a weak Protein A binding monoclonal antibody on MabSelect SuRe Protein A column.

FIG. 7 depicts the effect of a kosmotropic salt solution comprising various concentrations of ammonium sulfate on the dynamic binding capacity of a weak Protein A binding monoclonal antibody on MabSelect SuRe Protein A column with load titer 4.7-5.8 g/L.

FIG. 8 depicts the effect of a kosmotropic salt solution comprising various concentrations of ammonium sulfate on HCP levels in the MabSelect SuRe Protein A eluate for a weak Protein A binding monoclonal antibody. Load titer 4.7-5.8 g/L containing ˜200,000 ng/mg HCP.

FIG. 9 depicts the dynamic binding capacity (DBC) of canine MAb A on ProSep Ultra Plus Protein A resin in the absence and presence of kosmotropic salt.

### 4. DETAILED DESCRIPTION OF THE INVENTION

The present invention is directed to compositions and methods for purifying antibodies from a sample matrix. In certain embodiments, the methods are disclosed that are directed to improving Protein A performance for the purification of antibodies In particular, the present invention relates to compositions and methods for purifying antibodies exhibiting weak binding strength and low binding capacity for Protein A resin. In certain embodiments, the present invention is directed to enhancing the amount of an antibody of interest retained on a Protein A resin, where such antibody exhibits weak binding strength and low binding capacity for such resin. In certain embodiments, the present invention is also directed to enhance the binding capacity for antibodies such as chimeric, humanized or human antibodies which have high affinity for Protein A resin under standard Protein A capture operating conditions, as well as to enhance the separation of such antibodies from impurities, such as HCPs.

In certain embodiments, a kosmotropic salt solution, which contributes to the stability and structure of water-water interactions and causes water molecules to favorably interact with macromolecules such as proteins and also stabilizes the intermolecular interactions, is employed to promote the hydrophobic interaction between antibody and Protein A ligand thereby enhancing the retention of the antibody of interest on the Protein A resin. In certain embodiments, the concentration of the antibody of interest in a sample comprising the antibody of interest that is exposed to a Protein A resin is increased to enhance the retention of the antibody of interest on the Protein A resin. In certain embodiments, a combination of a kosmotropic salt solution and an increased concentration of the antibody of interest is employed to enhance the retention of the antibody of interest on the Protein A resin. In certain embodiments, a kosmotropic salt is employed to enhance the reduction of HCP levels in Protein A eluate for antibodies with high affinity for Protein A resin.

In certain embodiments, the purification strategies of the present invention may include one or more additional chromatography and/or filtration steps to achieve a desired degree of purification. For example, in certain embodiments, the chromatography step(s) can include one or more steps of ion exchange chromatography and/or hydrophobic interaction chromatography. In addition, in certain embodiments, the present invention is directed toward pharmaceutical compositions comprising one or more antibodies purified by methods described herein.

For clarity and not by way of limitation, this detailed description is divided into the following sub-portions:
4.1. Definitions;
4.2. Antibody Generation;
4.3. Antibody Production;
4.4. Antibody Purification;
4.5. Methods of Assaying Sample Purity;
4.6. Further Modifications; and
4.7. Pharmaceutical Compositions

### 4.1. Definitions

In order that the present invention may be more readily understood, certain terms are first defined.

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or “antibody portion”) includes fragments of an antibody that retain the ability to specifically bind to an antigen (e.g., hIL-12, hTNFα, or hIL-18). It has been shown that the antigen-binding function of an antibody can be performed by fragments of a full-length antibody. Examples of binding fragments encompassed within the term “antigen-binding portion” of an antibody include (i) a Fab fragment, a monovalent fragment comprising the VL, VH, CL and CH1 domains; (ii) a F(ab′)2 fragment, a bivalent fragment comprising two Fab fragments linked by a disulfide bridge at the hinge region; (iii) a Fd fragment comprising the VH and CH1 domains; (iv) a Fv fragment comprising the VL and VH domains of a single arm of an antibody, (v) a dAb fragment (Ward et al., (1989) Nature 341:544-546, the entire teaching of which is incorporated herein by reference), which comprises a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody may be part of a larger immunoadhesion molecule, formed by covalent or non-covalent association of the antibody with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding portions are complete domains or pairs of complete domains.

The terms “Kabat numbering” “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242, the entire teachings of which are incorporated herein by reference). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), e.g., in the CDRs and in particular CDR3. The mutations can be introduced using the “selective mutagenesis approach.” The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In one embodiment, these replacements are within the CDR regions. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor, L. D., et al. (1992) Nucl. Acids Res. 20:6287-6295, the entire teaching of which is incorporated herein by reference) or antibodies prepared, expressed, created or isolated by any other means that involves splicing of human immunoglobulin gene sequences to other DNA sequences. Such recombinant human antibodies have variable and constant regions derived from human germline immunoglobulin sequences (see, Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or back-mutation or both.

An “isolated antibody” includes an antibody that is substantially free of other antibodies having different antigenic specificities. Moreover, an isolated antibody may be substantially free of other cellular material and/or chemicals.

The term “Koff”, as used herein, is intended to refer to the off rate constant for dissociation of an antibody from the antibody/antigen complex.

The term “Kd”, as used herein, is intended to refer to the dissociation constant of a particular antibody-antigen interaction.

The phrase “dynamic binding capacity”, as used herein, is intended to refer to the amount of antibody that can bind to a chromatography media under flow conditions. This value is always lower than the static or saturation capacity.

The phrase “static binding capacity” as used herein, is intended to refer to the amount of target protein a column can bind if every available binding site is utilized. This is determined by loading a large excess of target protein either at very slow flow rates or after prolonged incubation in a closed system.

The phrase “weak binding strength” and “weak binding”, as used herein, is intended to refer to an antibody exhibiting a reduced binding capacity as compared to typical human IgG antibody, except for human IgG3 antibodies, e.g., such weak binding strength leads to about 2-10 fold lower binding capacity than that expected for a typical human IgG antibody, except for human IgG3 antibodies, for a particular chromatographic resin, e.g., a Protein A resin, and which would lead to inefficient purification under conventional purification conditions.

The phrase “nucleic acid molecule” includes DNA molecules and RNA molecules. A nucleic acid molecule may be single-stranded or double-stranded, but in one aspect is double-stranded DNA.

The phrase “isolated nucleic acid molecule,” as used herein in reference to nucleic acids encoding antibodies or antibody portions (e.g., VH, VL, CDR3), e.g. an antibody having a weak binding strength and low binding capacity for a Protein A resin. The phrase “isolated nucleic acid molecule” is also intended to include sequences encoding bivalent, bispecific antibodies, such as diabodies in which VH and VL regions contain no other sequences other than the sequences of the diabody.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “modifying”, as used herein, is intended to refer to changing one or more amino acids in the antibodies or antigen-binding portions thereof. The change can be produced by adding, substituting or deleting an amino acid at one or more positions. The change can be produced using known techniques, such as PCR mutagenesis.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

The phrase “contact position” includes an amino acid position in the CDR1, CDR2 or CDR3 of the heavy chain variable region or the light chain variable region of an antibody which is occupied by an amino acid that contacts antigen in one of the twenty-six known antibody-antigen structures. If a CDR amino acid in any of the twenty-six known solved structures of antibody-antigen complexes contacts the antigen, then that amino acid can be considered to occupy a contact position. Contact positions have a higher probability of being occupied by an amino acid which contact antigens than in a non-contact position. In one aspect, a contact position is a CDR position which contains an amino acid that contacts antigen in greater than 3 of the 26 structures (>1.5%). In another aspect, a contact position is a CDR position which contains an amino acid that contacts antigen in greater than 8 of the 25 structures (>32%).

### 4.2. Antibody Generation

The term “antibody” as used in this section refers to an intact antibody or an antigen binding fragment thereof.

The antibodies of the present disclosure can be generated by a variety of techniques, including immunization of an animal with the antigen of interest followed by conventional monoclonal antibody methodologies e.g., the standard somatic cell hybridization technique of Kohler and Milstein (1975) Nature 256: 495. Although somatic cell hybridization procedures are preferred, in principle, other techniques for producing monoclonal antibody can be employed e.g., viral or oncogenic transformation of B lymphocytes.

One animal system for preparing hybridomas is the murine system. Hybridoma production is a very well-established procedure. Immunization protocols and techniques for isolation of immunized splenocytes for fusion are known in the art. Fusion partners (e.g., murine myeloma cells) and fusion procedures are also known.

An antibody can be a canine, a feline, a horse, a human, a chimeric, or a humanized antibody. Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In certain non-limiting embodiments, the antibodies of this disclosure are those having a weak binding strength for Protein A. In certain embodiments, the antibodies are feline monoclonal antibodies. In certain embodiments, the antibodies are canine monoclonal antibodies. In certain embodiments, the antibodies are horse monoclonal antibodies. In other embodiments, the antibodies are human monoclonal antibodies. Such human monoclonal antibodies can be generated using transgenic or transchromosomic mice carrying parts of the human immune system rather than the mouse system. These transgenic and transchromosomic mice include mice referred to herein as the HuMAb Mouse® (Medarex, Inc.), KM Mouse® (Medarex, Inc.), and XenoMouse® (Amgen).

Moreover, alternative transchromosomic animal systems expressing human immunoglobulin genes are available in the art and can be used to raise antibodies of the disclosure. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise the antibodies of this disclosure.

In one embodiment, the antibodies of this disclosure are recombinant human antibodies, which can be isolated by screening of a recombinant combinatorial antibody library, e.g., a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612, the entire teachings of which are incorporated herein), examples of methods and reagents particularly amenable for use in generating and screening antibody display libraries can be found in, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982; the entire teachings of which are incorporated herein.

Human monoclonal antibodies of this disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

The antibodies or antigen-binding portions thereof, of this disclosure can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see, e.g., Canfield and Morrison (1991) J. Exp. Med. 173:1483-1491; and Lund et al. (1991) J. of Immunol. 147:2657-2662, the entire teachings of which are incorporated herein). Reduction in FeR binding ability of the antibody may also reduce other effector functions which rely on FcR interactions, such as opsonization and phagocytosis and antigen-dependent cellular cytotoxicity.

### 4.3. Antibody Production

To express an antibody of the invention, DNAs encoding partial or full-length light and heavy chains are inserted into one or more expression vector such that the genes are operatively linked to transcriptional and translational control sequences. (See, e.g., U.S. Pat. No. 6,914,128, the entire teaching of which is incorporated herein by reference.) In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into a separate vector or, more typically, both genes are inserted into the same expression vector. The antibody genes are inserted into an expression vector by standard methods (e.g., ligation of complementary restriction sites on the antibody gene fragment and vector, or blunt end ligation if no restriction sites are present). Prior to insertion of the antibody or antibody-related light or heavy chain sequences, the expression vector may already carry antibody constant region sequences. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, a recombinant expression vector of the invention can carry one or more regulatory sequence that controls the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, e.g., in Goeddel; Gene Expression Technology: Methods in Enzymology 185, Academic Press, San Diego, Calif. (1990), the entire teaching of which is incorporated herein by reference. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al., the entire teachings of which are incorporated herein by reference.

In addition to the antibody chain genes and regulatory sequences, a recombinant expression vector of the invention may carry one or more additional sequences, such as a sequence that regulates replication of the vector in host cells (e.g., origins of replication) and/or a selectable marker gene. The selectable marker gene facilitates selection of host cells into which the vector has been introduced (see e.g., U.S. Pat. Nos. 4,399,216, 4,634,665 and 5,179,017, all by Axel et al., the entire teachings of which are incorporated herein by reference). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in dhfr-host cells with methotrexate selection/amplification) and the neo gene (for G418 selection).

An antibody of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. Nos. 4,816,397 & 6,914,128, the entire teachings of which are incorporated herein.

For expression of the light and heavy chains, the expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood (1985) Immunology Today 6:12-13, the entire teaching of which is incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Suitable mammalian host cells for expressing the recombinant antibodies of the invention include Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N.Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F10™ (Sigma), Minimal Essential Medium™ ((MEM), (Sigma), RPMI-1640 (Sigma), and Dulbecco's Modified Eagle's Medium™ ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. No. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Host cells can also be used to produce portions of intact antibodies, such as Fab fragments or scFv molecules. It is understood that variations on the above procedure are within the scope of the present invention. For example, in certain embodiments it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to the antigen to which the putative antibody of interest binds. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the one to which the putative antibody of interest binds, depending on the specificity of the antibody of the invention, by crosslinking an antibody of the invention to a second antibody by standard chemical crosslinking methods.

In a suitable system for recombinant expression of an antibody of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

When using recombinant techniques, the antibody can be produced intracellularly, in the periplasmic space, or directly secreted into the medium. In one aspect, if the antibody is produced intracellularly, as a first step, the particulate debris, either host cells or lysed cells (e.g., resulting from homogenization), can be removed, e.g., by centrifugation or ultrafiltration. Where the antibody is secreted into the medium, supernatants from such expression systems can be first concentrated using a commercially available protein concentration filter, e.g., an Amicon™ or Millipore Pellicon™ ultrafiltration unit.

Prior to the process of the invention, procedures for purification of antibodies from cell debris initially depend on the site of expression of the antibody. Some antibodies can be secreted directly from the cell into the surrounding growth media; others are made intracellularly. For the latter antibodies, the first step of a purification process typically involves: lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon™ or Millipore Pellicon™ ultrafiltration unit. Where the antibody is secreted into the medium, the recombinant host cells can also be separated from the cell culture medium, e.g., by tangential flow filtration. Antibodies can be further recovered from the culture medium using the antibody purification methods of the invention.

### 4.4. Antibody Purification

### 4.4.1 Antibody Purification Generally

The invention provides a method for producing a purified (or “HCP-reduced”) antibody preparation from a mixture comprising an antibody and at least one HCP. The purification process of the invention begins at the separation step when the antibody has been produced using methods described above and conventional methods in the art. Table 1 summarizes one embodiment of a purification scheme. Variations of this scheme, including, but not limited to, where the order of the ion exchange and HIC steps is reversed, or where the order of viral inactivation and protein A steps is reversed, or removal of the pre-capture ultrafiltration step, are envisaged and are within the scope of this invention.

TABLE 1
Purification steps with their associated purpose.

| Purification step | Purpose |
|---|---|
| Primary recovery | Clarification of cell culture sample matrix by |
| (Centrifugation and/ | removing cells and cell debris |
| or Depth filtration) | |
| Ultrafiltration | Concentrating antibody |
| Viral inactivation | Inactivation of encapsulated virus by detergent or |
| | low pH |
| Protein A Affinity | Antibody capture, host cell protein and associated |
| chromatography | impurity reduction |
| Depth filtration | Remove turbidity/precipitates and impurities |
| Ion exchange | Reduction of host cell proteins, DNA, aggregates, |
| chromatography | leached protein A and virus |
| (anion or cation) | |
| Hydrophobic | Reduction of antibody aggregates, host cell |
| interaction | proteins, DNA, leached protein A and virus |
| chromatography | |
| Viral filtration | Removal of virus, if present |
| Ultrafiltration/ | Concentrate and formulate antibody |
| Diafiltration | |

Once a clarified solution or mixture comprising the antibody has been obtained, separation of the antibody from the other proteins produced by the cell, such as HCPs, is performed using Protein A affinity chromatography and, in certain embodiments, a combination of one or more different purification techniques, including ion exchange separation step(s) and hydrophobic interaction separation step(s). Such additional purification steps separate mixtures of proteins on the basis of their charge, degree of hydrophobicity, and/or size. In one aspect of the invention, such additional separation steps are performed using chromatography, including hydrophobic, anionic or cationic interaction. Several different chromatography resins are available for each of these techniques, allowing accurate tailoring of the purification scheme to the particular protein involved. The essence of each of the separation methods is that proteins can either traverse at different rates down a column, achieving a physical separation that increases as they pass further down the column, or to adhere selectively to the separation medium, being then differentially eluted by different solvents. In some cases, the antibody is separated from impurities when the impurities specifically adhere to the column and the antibody does not, i.e., the antibody is present in the flow-through.

### 4.4.2 Primary Recovery

In certain embodiments, the initial steps of the purification methods of the present invention involve the clarification and primary recovery of antibody from a sample matrix. In certain embodiments, the primary recovery will include one or more centrifugation steps to separate the antibody product from the cells and cell debris. Centrifugation of the sample can be run at, for example, but not by way of limitation, 7,000×g to approximately 12,750×g. In the context of large scale purification, such centrifugation can occur on-line with a flow rate set to achieve, for example, but not by way of limitation, a turbidity level of 150 NTU in the resulting supernatant. Such supernatant can then be collected for further purification, or in-line filtered through one or more depth filters for further clarification of the sample.

In certain embodiments, the primary recovery will include the use of one or more depth filtration steps only to clarify the sample matrix and thereby aid in purifying the antibodies of interest in the present invention. In other embodiments, the primary recovery will include the use of one or more depth filtration steps post centrifugation to further clarify the sample matrix. Depth filters contain filtration media having a graded density. Such graded density allows larger particles to be trapped near the surface of the filter while smaller particles penetrate the larger open areas at the surface of the filter, only to be trapped in the smaller openings nearer to the center of the filter. In certain embodiments the depth filtration step can use Millistak+ X0HC Pod depth filter. Although certain embodiments employ depth filtration steps only during the primary recovery phase, other embodiments employ depth filters during one or more additional phases of purification. Non-limiting examples of depth filters that can be used in the context of the instant invention include the Millistak+ F0HC, D0HC, A1HC, B1HC depth filters (EMD Millipore), Cuno™ model 30/60ZA, 60/90 ZA, VR05, VR07, delipid depth filters (3M Corp.). A 0.2 μm filter such as Sartorius's 0.45/0.2 μm Sartopore™ bi-layer or Millipore's Express SHR or SHC filter cartridges typically follows the depth filters.

In certain embodiments, the primary recovery process can also be a point at which to reduce or inactivate viruses that can be present in the sample matrix. For example, any one or more of a variety of methods of viral reduction/inactivation can be used during the primary recovery phase of purification including heat inactivation (pasteurization), pH inactivation, solvent/detergent treatment, UV and γ-ray irradiation and the addition of certain chemical inactivating agents such as β-propiolactone or e.g., copper phenanthroline as in U.S. Pat. No. 4,534,972, the entire teaching of which is incorporated herein by reference. In certain embodiments of the present invention, the sample matrix is exposed to detergent viral inactivation during the primary recovery phase. In other embodiments, the sample matrix may be exposed to low pH inactivation during the primary recovery phase.

Methods of detergent viral inactivation can include, but are not limited to, incubating the mixture for a period of time in the presence of detergent such as Tween 20, Tween 80, and Triton X-100. In certain embodiments the detergent concentration may range from 0.00001% (v/v) to 2% (v/v), or, in certain embodiments, in the range of 0.0001% (v/v) to 0.5% (v/v), or, in certain embodiments, in the range of 0.005% (v/v) to 0.1% (v/v), or, in certain embodiments, at about 0.1% (v/v). The choice of detergent level largely depends on the stability profile of the antibody product at the selected conditions. It is known that the quality of the target antibody during detergent viral inactivation can be affected by detergent concentration and the duration of the detergent incubation. In certain embodiments, the duration of the detergent incubation will be from 0.5 hr to 4 hr, in certain embodiments it will be 0.5 hr to 2 hr, and in certain embodiments the duration will be 1 hr. Virus inactivation is dependent on these same parameters in addition to protein concentration, which may limit inactivation efficiency at high concentrations. Thus, the proper parameters of protein concentration, detergent concentration and duration of inactivation can be selected to achieve the desired level of viral inactivation.

Methods of pH viral inactivation can include, but are not limited to, incubating the mixture for a period of time at low pH, and subsequently neutralizing the pH and removing particulates by filtration. In certain embodiments the mixture will be incubated at a pH of between about 2 and 5, or, in certain embodiments, at a pH of between about 3 and 4, or, in certain embodiments, at a pH of about 3.5. The pH of the sample mixture may be lowered by any suitable acid including, but not limited to, phosphoric acid, citric acid, acetic acid, caprylic acid, or other suitable acids. The choice of pH level largely depends on the stability profile of the antibody product and buffer components. It is known that the quality of the target antibody during low pH virus inactivation is affected by pH and the duration of the low pH incubation. In certain embodiments the duration of the low pH incubation will be from 0.5 hr to 2 hr, in certain embodiments it will be 0.5 hr to 1.5 hr, and in certain embodiments the duration will be 1 hr. Virus inactivation is dependent on these same parameters in addition to protein concentration, which may limit inactivation at high concentrations. Thus, the proper parameters of protein concentration, pH, and duration of inactivation can be selected to achieve the desired level of viral inactivation.

In those embodiments where viral reduction/inactivation is employed, the sample mixture can be adjusted, as needed, for further purification steps. For example, following low pH viral inactivation, the pH of the sample mixture is typically adjusted to a more neutral pH, e.g., from about 4.5 to about 8.5, prior to continuing the purification process. Additionally, the mixture may be diluted with water for injection (WFI) to obtain a desired conductivity.

### 4.4.3 Protein A Affinity Chromatography

In certain embodiments, the primary recovery sample is subjected to Protein A affinity chromatography to purify the antibody of interest away from HCPs. There are several commercial sources for Protein A resin. One suitable resin is MabSelect SuRe™ from GE Healthcare. A non-limiting example of a suitable column packed with MabSelect SuRe™ is an about 1.0 cm diameter×about 22 cm long column (˜17 mL bed volume). This size column can be used for small scale purifications and can be compared with other columns used for scale ups. For example, a 20 cm×22 cm column whose bed volume is about 6.9 L can be used for larger purifications. Regardless of the column, the column can be packed using a suitable resin such as MabSelect SuRe™, MabSelect SuRe LX, MabSelect, MabSelect Xtra, rProtein A Sepharose from GE Healthcare, and ProSep HC, ProSep Ultra, and ProSep Ultra Plus from EMD Millipore.

In certain embodiments it will be advantageous to determine the dynamic binding capacity (DBC) of the Protein A resin in order to tailor the purification to the particular antibody of interest. For example, but not by way of limitation, the DBC of a MabSelect SuRe™ column can be determined either by a single flow rate load or dual-flow load strategy. The single flow rate load can be evaluated at a velocity of about 335 cm/hr throughout the entire loading period. The dual-flow rate load strategy can be determined by loading the column up to about 24 mg protein/mL resin at a linear velocity of about 335 cm/hr, then reducing the linear velocity to 220 cm/hr to allow longer residence time for the last portion of the load.

In certain embodiments, the Protein A column can be equilibrated with a suitable buffer prior to sample loading. A non-limiting example of a suitable buffer is a Tris buffer with or without kosmotropic salt, pH of about 7.5. A non-limiting example of suitable equilibration conditions is 20 mM Tris, pH of about 7.5, a PBS buffer, or a 20 mM Tris, 1.1 M ammonium sulfate, pH 7.5 buffer. Following this equilibration, the sample can be loaded onto the column. Following the loading of the column, the column can be washed one or multiple times using, e.g., the equilibrating buffer. Other washes, including washes employing different buffers, can be employed prior to eluting the column. For example, the column can be washed using one or more column volumes of 20 mM Tris, pH 7.5 with lower level of salt (e.g. 0.6 M ammonium sulfate) than that in the equilibration buffer. This wash can optionally be followed by one or more washes using the equilibrating buffer. The Protein A column can then be eluted using an appropriate elution buffer. A non-limiting example of a suitable elution buffer is an acetic acid/NaCl buffer, pH of about 3.5, or a Tris buffer with pH of about 8.5. Suitable conditions are, e.g., 0.1 M acetic acid, pH of about 3.5, or 20 mM Tris, pH of about 8.5. The eluate can be monitored using techniques well known to those skilled in the art. For example, the absorbance at OD₂₈₀ can be followed. Column eluate can be collected starting with an initial deflection of about 0.5 AU to a reading of about 0.5 AU at the trailing edge of the elution peak. The elution fraction(s) of interest can then be prepared for further processing. For example, the collected sample can be titrated to a pH in the range of 5 to 8 using Tris buffer (e.g., 1.0 M) at a pH of about 10, and/or diluted to obtain a lower conductivity sample. Optionally, this titrated sample can be filtered and further processed.

In certain embodiments, the Protein A column can be equilibrated with PBS buffer or a pH 7.5 Tris buffer prior to sample loading. Following the loading phase, the column is washed with the equilibration buffer, or followed by equilibration and other wash buffers and then equilibration buffer again. The Protein A column can then be eluted using an appropriate high pH buffer. A non-limiting example of a suitable elution buffer is a 20 mM Tris, pH 8.5 buffer. The eluate can be collected based on UV280 elution profile from the peak front reading of 0.5 AU to the peak tail reading of 0.5 AU. The elution fraction(s) of interest can then be prepared for further processing.

In certain embodiments, a kosmotropic salt solution is supplemented into the sample matrix comprising the antibodies of interest to form a conditioned clarified harvest sample prior to contacting with a Protein A resin. The kosmotropic salt solution comprises at least one kosmotropic salt. Examples of suitable kosmotropic salts include, but are not limited to ammonium sulfate, sodium sulfate, sodium citrate, potassium sulfate, potassium phosphate, sodium phosphate, and a combination thereof. In one aspect, the kosmotropic salt is ammonium sulfate; in another aspect, the kosmotropic salt is sodium sulfate; and in another aspect, the kosmotropic salt is sodium citrate. The kosmotropic salt is present in the kosmotropic salt solution at a concentration of from about 0.3 M to about 1.1 M. In one embodiment, the kosmotropic salt is present in the kosmotropic salt solution at a concentration of about 0.5 M.

In certain embodiments, an increased concentration of the antibody of interest as compared to conventional purification strategies is loaded onto to a Protein A resin. For antibodies with relatively low binding capacity for the Protein A resin, such an increased load concentration of the antibody of interest enhances its binding capacity to the Protein A resin. In certain of such embodiments, the antibody in the sample matrix that is contacted to a Protein A resin has a concentration of from about 1 g/L to about 10 g/L. In certain embodiments the concentration is from about 1.5 g/L to about 8 g/L, about 1.5 g/L to about 5.8 g/L, about 1.7 g/L to about 5.8 g/L, about 1.9 g/L to about 5.45 g/L, about 1.9 g/L to about 4.95 g/L, about 1.9 g/L to about 4.7 g/L, about 1.9 g/L to about 4.5 g/L, or about 1.9 g/L to about 3.6 g/L. In certain embodiments, the concentration is about 1.5 g/L, about 1.9 g/L, about 3.6 g/L, about 4.5 g/L, about 4.7 g/L, about 4.95 g/L, about 5.45 g/L, or about 5.8 g/L.

In certain embodiments, a primary recovery sample matrix comprising the antibodies of interest or antigen-binding portions thereof, is subject to ultrafiltration first to enrich the antibody in the matrix and then supplemented with a kosmotropic salt solution to form a conditioned primary recovery sample. This high concentration of the antibody harvest sample is then loaded to a Protein A column. The concentration of the kosmotropic salt present in such conditioned harvest sample ranges from about 0.3 M to about 1.1 M. In one embodiment, the kosmotropic salt is present in the kosmotropic salt solution at a concentration of about 0.5 M. The antibody in the primary recovery sample that is contacted with a Protein A resin has a concentration of from about 1 g/L to about 10 g/L. In certain embodiments the concentration is from about 1.5 g/L to about 8 g/L, about 1.5 g/L to about 5.8 g/L, about 1.7 g/L to about 5.8 g/L, about 1.9 g/L to about 5.45 g/L, about 1.9 g/L to about 4.95 g/L, about 1.9 g/L to about 4.7 g/L, about 1.9 g/L to about 4.5 g/L, or about 1.9 g/L to about 3.6 g/L. In certain embodiments, the concentration is about 1.5 g/L, about 1.9 g/L, about 3.6 g/L, about 4.5 g/L, about 4.7 g/L, about 4.95 g/L, about 5.45 g/L, or about 5.8 g/L.

The Protein A eluate may subject to a viral inactivation step either by detergent or low pH, provided this step is not performed prior to the Protein A capture operation. A proper detergent concentration or pH and time can be selected to obtain desired viral inactivation results. After viral inactivation, the Protein A eluate is usually pH and/or conductivity adjusted for the following purification steps.

The Protein A eluate may subject to filtration through a depth filter to remove turbidity and/or various impurities from the antibody of interest prior to additional chromatography polishing steps. Examples of depth filters include, but not limited to, Millistak+ X0HC, F0HC, D0HC, A1HC, and B1HC Pod filters (EMD Millipore), or Zeta Plus 30ZA/60ZA, 60ZA/90ZA, delipid, VR07, and VR05 filters (3M). In one embodiment, X0HC depth filter can be used to process the Protein A eluate before ion-exchange chromatography step. The Protein A eluate pool may need to be conditioned to proper pH and conductivity to obtain desired impurity removal and product recovery from the depth filtration step.

### 4.4.4 Ion Exchange Chromatography

In certain embodiments, the instant invention provides methods for producing a HCP-reduced antibody preparation from a mixture comprising an antibody and at least one HCP by subjecting the mixture to at least one ion exchange separation step after the above-described Protein A affinity chromatographic step, such that an eluate comprising the antibody is obtained. Ion exchange separation includes any method by which two substances are separated based on the difference in their respective ionic charges, and can employ either cationic exchange material or anionic exchange material.

The use of a cationic exchange material versus an anionic exchange material is based on the overall charge of the protein at a given solution condition. Therefore, it is within the scope of this invention to employ an anionic exchange step prior to the use of a cationic exchange step, or a cationic exchange step prior to the use of an anionic exchange step. Furthermore, it is within the scope of this invention to employ only a cationic exchange step, only an anionic exchange step, or any serial combination of the two.

In performing the separation, the initial antibody mixture can be contacted with the ion exchange material by using any of a variety of techniques, e.g., using a batch purification technique or a chromatographic technique.

For example, in the context of batch purification, ion exchange material is prepared in, or equilibrated to, the desired starting buffer. Upon preparation, or equilibration, a slurry of the ion exchange material is obtained. The antibody solution is contacted with the slurry to adsorb the antibody to be separated to the ion exchange material. The solution comprising the HCP(s) that do not bind to the ion exchange material is separated from the slurry, e.g., by allowing the slurry to settle and removing the supernatant. The slurry can be subjected to one or more wash steps. If desired, the slurry can be contacted with a solution of higher conductivity to desorb HCPs that have bound to the ion exchange material. In order to elute bound polypeptides, the salt concentration of the buffer can be increased.

Ion exchange chromatography may also be used as an ion exchange separation technique. Ion exchange chromatography separates molecules based on differences between the overall charge of the molecules. For the purification of an antibody, the antibody must have a charge opposite to that of the functional group attached to the ion exchange material, e.g., resin, in order to bind. For example, antibodies, which generally have an overall positive charge in the buffer pH below its pI, will bind well to cation exchange material, which contain negatively charged functional groups.

A packed ion-exchange chromatography column or an ion-exchange membrane device can be operated either in antibody bind-elute mode or flow-through mode. In the bind-elute mode, the column or the membrane device is first conditioned with a buffer with low ionic strength and proper pH under which the protein carries sufficient opposite charge to that immobilized on the resin based matrix. During the feed load, the protein of interest will be adsorbed to the resin due to electrostatic attraction. After washing the column or the membrane device with the equilibration buffer or another buffer with different pH and/or conductivity, the product recovery is achieved by increasing the ionic strength (i.e., conductivity) of the elution buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution). In the flow-through mode, the column or the membrane device is operated at selected pH and conductivity such that the protein of interest does not bind to the resin or the membrane while the impurities such as HCP, aggregates, DNA and virus will be retained to the column or the membrane. The column is then regenerated before next use.

Anionic or cationic substituents may be attached to matrices in order to form anionic or cationic supports for chromatography. Non-limiting examples of anionic exchange substituents include diethylaminoethyl (DEAE), quaternary aminoethyl (QAE) and quaternary amine (Q) groups. Cationic substitutents include carboxymethyl (CM), sulfoethyl (SE), sulfopropyl (SP), phosphate (P) and sulfonate (S). Cellulose ion exchange resins such as DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and S-SEPHAROSE® and SEPHAROSE® Fast Flow are all available from GE Healthcare. Further, both DEAE and CM derivitized ethylene glycol-methacrylate copolymer such as TOYOPEARL™ DEAE-650S or M and TOYOPEARL™ CM-650S or M are available from Toso Haas Co., Philadelphia, Pa.

A mixture comprising an antibody and impurities, e.g., HCP(s), is loaded onto an ion exchange column, such as an anion exchange column. For example, but not by way of limitation, the mixture can be loaded at a load level of about 40 g protein/L resin depending upon the column used. An example of a suitable anion exchange resin is Capto Q (GE Healthcare). The mixture loaded onto Capto Q column can be subsequently washed with wash buffer (equilibration buffer). The antibody is then eluted from the column, and a first eluate is obtained.

This ion exchange step facilitates the purification of the antibody of interest by reducing impurities such as HCPs, DNA and aggregates. In certain aspects, the ion exchange column is an anion exchange column. For example, but not by way of limitation, a suitable resin for such an anion exchange column is Capto Q, Q Sepharose Fast Flow, and Poros HQ 50. These resins are available from commercial sources such as GE Healthcare and Life Technologies. This anion exchange chromatography process can be carried out at or around room temperature.

### 4.4.5 Hydrophobic Interaction Chromatography

The present invention also features methods for producing a HCP-reduced antibody preparation from a mixture comprising an antibody and at least one HCP further comprising a hydrophobic interaction chromatography (HIC) step subsequent to the Protein A affinity capture step and followed by an ion-exchange chromatography step. When such a HIC step is employed, the post-protein A depth filtration may not be needed provided that the turbidity of the conditioned protein A eluate is sufficiently low such that no clogging of the HIC column would occur. The eluate generated from this step, such as those disclosed herein, has reduced levels of HCPs, DNA, aggregates, or leached protein A.

In performing the separation, the sample mixture is contacted with the HIC material, e.g., using a batch purification technique or using a column or membrane chromatography. Prior to HIC purification it may be desirable to adjust the concentration of the kosmotropic salt to achieve desired protein binding to the resin or the membrane.

For example, in the context of batch purification, HIC material is prepared in or equilibrated with a desired equilibration buffer. A slurry of the HIC material is obtained. The antibody solution is contacted with the slurry to allow antibody adsorption to the HIC material. The solution comprising the HCPs that do not bind to the HIC material is separated from the slurry, e.g., by allowing the slurry to settle and removing the supernatant. The slurry can be subjected to one or more washing steps. If desired, the slurry can be contacted with a solution of lower conductivity to desorb antibodies that have bound to the HIC material. In order to elute bound antibodies, the salt concentration can be decreased

Whereas ion exchange chromatography relies on the charge of the antibodies to isolate them, hydrophobic interaction chromatography employs the hydrophobic properties of the antibodies. Hydrophobic groups on the antibody interact with hydrophobic groups of the resin or the membrane. The more hydrophobic a protein is the stronger it will interact with the column or the membrane. Thus the HIC step removes host cell derived impurities (e.g., DNA and other high and low molecular weight product-related species).

Like ion exchange chromatography, a HIC column or membrane device can also be operated in either product bind-elute or flow-through mode. The bind-elute mode of operation has been explained above. For flow-through, the protein sample typically contains a relatively low level of komotropic salt than that used in the bind-elute mode. During this loading process, impurities such as HCP and aggregates will bind to the resin while product flows through the column. After loading, the column is washed with a buffer and then regenerated with water and cleaned with caustic solution to remove the bound impurities before next use. For example, the antibody of the present invention coming out of the kosmotropic salt-assisted Protein A capture step can be flowed through a HIC column (e.g. Capto Phenyl column) after proper conductivity adjustment of the Protein A eluate.

Hydrophobic interactions are strongest at high ionic strength, therefore, this form of separation is conveniently performed following a low salt elution step which are typically used in an ion exchange chromatography. Adsorption of the antibody to a HIC column is favored by high salt concentrations, but the actual concentrations can vary over a wide range depending on the nature of the antibody, salt type and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba²⁺; Ca²⁺; Mg²⁺; Li⁺; Cs⁺; Na⁺; K⁺; Rb⁺; NH₄⁺, while anions may be ranked in terms of increasing chaotropic effect as PO₄³⁻; SO₄²⁻; CH₃CO₃⁻; Cl⁻; Br⁻; NO₃⁻; CO₄⁻; I⁻; SCN⁻.

In general, Na⁺, K⁺ or NH₄⁺ sulfates effectively promote ligand-protein interaction in HIC. Salts may be formulated that influence the strength of the interaction as given by the following relationship: (NH₄)₂SO₄>Na₂SO₄>NaCl>NH₄Cl>NaBr>NaSCN. In general, salt concentrations of between about 0.75 and about 2 M ammonium sulfate or between about 1 and 4 M NaCl are useful.

HIC media normally comprise a base matrix (e.g., cross-linked agarose or synthetic copolymer material) to which hydrophobic ligands (e.g., alkyl or aryl groups) are coupled. A suitable HIC media comprises an agarose resin or a membrane functionalized with phenyl groups (e.g., a Phenyl Sepharose™ from GE Healthcare or a Phenyl Membrane from Sartorius). Many HIC resins are available commercially. Examples include, but are not limited to, Capto Phenyl, Phenyl Sepharose™ 6 Fast Flow with low or high substitution, Phenyl Sepharose™ High Performance, Octyl Sepharose™ High Performance (GE Healthcare); Fractogel™ EMD Propyl or Fractogel™ EMD Phenyl (E. Merck, Germany); Macro-Prep™ Methyl or Macro-Prep™ t-Butyl columns (Bio-Rad, California); WP HI-Propyl (C3)™ (J. T. Baker, New Jersey); and Toyopearl™ ether, phenyl or butyl (TosoHaas, Pa.).

### 4.4.6 Viral Filtration

Viral filtration is a dedicated viral reduction step in the entire purification process. This step is usually performed post chromatographic polishing steps. Viral reduction can be achieved via the use of suitable filters including, but not limited to, Planova 20N™, 50 N or BioEx from Asahi Kasei Pharma, Viresolve™ filters from EMD Millipore, ViroSart CPV from Sartorius, or Ultipor DV20 or DV50™ filter from Pall Corporation. It will be apparent to one of ordinary skill in the art to select a suitable filter to obtain desired filtration performance.

### 4.4.7 Ultrafiltration/Diaffiltration

Certain embodiments of the present invention employ ultrafiltration and diafiltration steps to further concentrate and formulate the antibody product. Ultrafiltration is described in detail in: Microfiltration and Ultrafiltration: Principles and Applications, L. Zeman and A. Zydney (Marcel Dekker, Inc., New York, N.Y., 1996); and in: Ultrafiltration Handbook, Munir Cheryan (Technomic Publishing, 1986; ISBN No. 87762-456-9). One filtration process is Tangential Flow Filtration as described in the Millipore catalogue entitled “Pharmaceutical Process Filtration Catalogue” pp. 177-202 (Bedford, Mass., 1995/96). Ultrafiltration is generally considered to mean filtration using filters with a pore size of smaller than 0.1 μm. By employing filters having such small pore size, the volume of the sample can be reduced through permeation of the sample buffer through the filter membrane pores while antibodies are retained above the membrane surface.

Diafiltration is a method of using membrane filters to remove and exchange salts, sugars, and non-aqueous solvents, to separate free from bound species, to remove low molecular-weight species, and/or to cause the rapid change of ionic and/or pH environments. Microsolutes are removed most efficiently by adding solvent to the solution being diafiltered at a rate approximately equal to the permeate flow rate. This washes away microspecies from the solution at a constant volume, effectively purifying the retained antibody. In certain embodiments of the present invention, a diafiltration step is employed to exchange the various buffers used in connection with the instant invention, optionally prior to further chromatography or other purification steps, as well as to remove impurities from the antibody preparations.

One of ordinary skill in the art can select appropriate membrane filter device for the UF/DF operation. Examples of membrane cassettes suitable for the present invention include, but not limited to, Pellicon 2 or Pellicon 3 cassettes with 10 kD, 30 kD or 50 kD membranes from EMD Millipore, Kvick 10 kD, 30 kD or 50 kD membrane cassettes from GE Healthcare, and Centramate or Centrasette 10 kD, 30 kD or 50 kD cassettes from Pall Corporation.

### 4.4.8 Exemplary Purification Strategies

Multiple process schemes based on the concepts of present invention can be employed to efficiently purify a MAb with weak binding strength for a Protein A chromatography media. Two non-limiting examples are described here for illustration purposes. Variation and modification of these examples, such as changing the order of one or more of the steps, are within the scope of this invention.

### 4.4.8.1. A Two-Column Purification Scheme

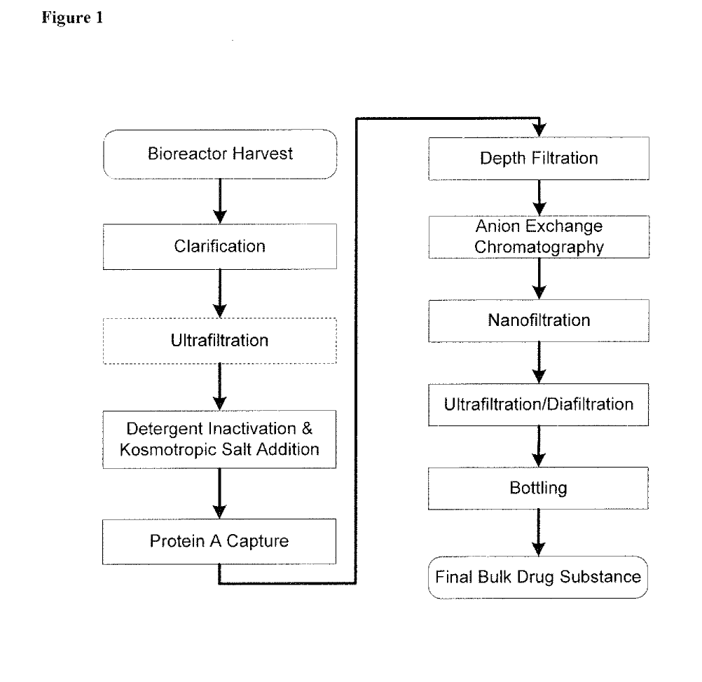

FIG. 1 depicts a two-column process for purification of a weak Protein A binding MAb. The harvest sample is first clarified to remove cells and cell debris using centrifugation, depth filtration, or the combination of both. If the clarified harvest, also known as the “primary recovery sample,” has an MAb titer less than about 1 g/L, it can be concentrated first by an ultrafiltration step to increase MAb concentration prior to further processing. The ultrafiltration is typically operated in the tangential flow filtration (or TFF) mode. The concentrated harvest can then be added with a detergent (e.g. 0.1% Tween 80 or Triton-X 100) to inactivate mammalian virus if present. The inactivated primary recovery harvest sample is then supplemented with a kosmotropic salt to obtain a conditioned primary recovery harvest sample with desired salt and protein concentration. The kosmotropic salt can be (NH₄)₂SO₄, Na₂SO₄, NaCitrate, K₂SO₄, K₃PO₄, Na₃PO₄, or a combination thereof. The MAb concentration in this conditioned primary recovery sample can range from about 1 g/L to about 10 g/L, while in certain embodiments the concentration is from about 1.5 g/L to about 8 g/L, about 1.5 g/L to about 5.8 g/L, about 1.7 g/L to about 5.8 g/L, about 1.9 g/L to about 5.45 g/L, about 1.9 g/L to about 4.95 g/L, about 1.9 g/L to about 4.7 g/L, about 1.9 g/L to about 4.5 g/L, or about 1.9 g/L to about 3.6 g/L. In certain embodiments, the concentration is about 1.5 g/L, about 1.9 g/L, about 3.6 g/L, about 4.5 g/L, about 4.7 g/L, about 4.95 g/L, about 5.45 g/L, or about 5.8 g/L. This material is usually filtered through a 0.2 um filter to remove any precipitates or turbidity formed during this process.

The conditioned and filtered primary recovery harvest sample is then subjected to a Protein A capture chromatography step. Any commercial Protein A resins or membranes can be employed here, including but not limited to, MabSelect SuRe, MabSelect SuRe LX, MabSelect, MabSelect Xtra from GE Healthcare, and ProSep HC, ProSep Ultra Plus, and ProSep Ultra Plus from EMD Millipore. The equilibration buffer contains the same concentrations of the komotropic salt as that used in the load material. One or multiple wash steps can be performed to reduce impurities such as HCPs. These wash buffers may contain the same concentrations of komotropic salt as used in the load, or higher or lower concentrations. In certain embodiments, a higher salt buffer was used in the first wash step followed by the equilibration buffer wash. An example of a suitable equilibration buffer is a Tris buffer with pH of about 6 to 8, or, in certain embodiments, about 7.5, containing a komotropic salt. A specific example of suitable equilibration is 20 mM Tris, 0.5 M (NH₄)₂SO₄, pH 7.5, wash 1 buffer is 20 mM Tris, 0.8 M (NH₄)₂SO₄, pH 7.5, and wash 2 buffer is the same as equilibration buffer. The Protein A column elution can be achieved using either a low pH or a high pH buffer. An example of high pH buffer is 20 mM Tris, pH 8.5 buffer. The eluate can be monitored using techniques well known to those skilled in the art. For example, the absorbance at UV₂₈₀ can be followed. The eluate can be collected starting with an initial deflection of about 500 mAU to a reading of about 500 mAU at the trailing edge of the elution peak. The elution fraction(s) of interest can then be prepared for further processing.

The Protein A eluate can be pH and/or conductivity adjusted to a target condition prior to fine purification. An example of such condition is pH 8 and about 28 mS/cm. A depth filtration step can be used to remove any precipitate or turbidity formed during this conditioning step; it also reduces impurities including HCP, aggregates, DNA, and leach Protein A. In certain embodiments, the depth filter is Millistak+X0HC Pod filter (EMD Millipore). Other filters with cationic charge functionality can also be used in this step.

The depth filtrate can then be purified through an anion exchange (AEX) chromatography step to further remove various impurities. Either AEX resin or AEX membrane can be used for this operation. An example of AEX resin is Capto Q or Q Sepharose Fast Flow (GE Healthcare). Either bind-elute or flow-through mode can be used for this step. In certain embodiments, Capto Q column was operated in the bind-elute mode to achieve desired product purity.

The AEX eluate is then processed through a viral filtration step to ensure sufficient viral removal for the overall process. Selecting a suitable viral filter can be performed by anyone skilled in the art. An example of suitable viral filter is Planova 20 N or BioEx from Asahi.

The viral filtrate is subjected to final ultrafiltration and diafiltration to formulate the antibody product. Commercial filters are available to effectuate this step. For example, a Biomax 30 kD membrane cassette (EMD Millipore) can be used to complete this step. The final product is then filled into proper containers before storage.

### 4.4.8.2. A Three-Column Purification Scheme

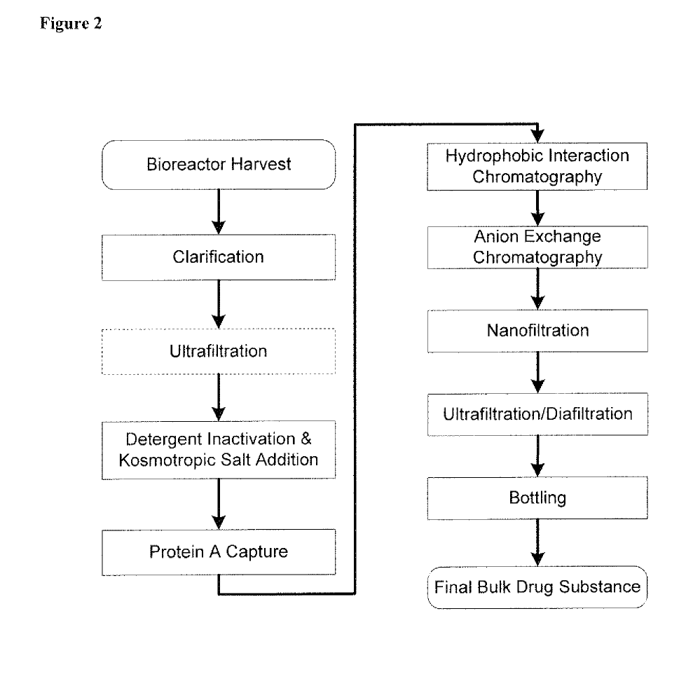

FIG. 2 shows a three-column process for purification of a weak Protein A binding MAb molecule. The key difference between this process and the two-column process is that a HIC chromatography step is used prior to the AEX polishing. When there is no significant precipitate or turbidity in the conditioned Protein A eluate, it can be processed directly through a HIC step first to remove HCP, DNA, aggregates and leached Protein A. This HIC step can be run in either flow-through or bind-elute mode, and can be a resin or a membrane. In some embodiments, Capto Phenyl resin is used and is run in the flow-through mode (GE Healthcare). The column is equilibrated with 20 mM Tris, 0.1 M (NH₄)₂SO₄, pH 7.5 buffer, then loaded with conditioned Protein A eluate at pH 7.5 and conductivity ˜23 mS/cm, and finally washed with the equilibration buffer again to recover the residual product retained within the column. The column may be loaded to 80 g/L of antibody, and the flow-through pool is collected during the load when UV280 reading reached 200 mAU and stopped during the wash when UV280 reading dropped back to 200 mAU. The HIC eluate is then processed through AEX chromatography to further purify the antibody to desired final purity. All the other steps are similar to those described in the two-column process scheme.

In the case of significant precipitate or turbidity is formed during the conditioning of the Protein A eluate, a depth filtration step can be used before the HIC chromatography. In this case, any depth filter that can remove particulates may be employed here.

In addition to the two exemplary process schemes described above, the cation exchange chromatography (CEX) step can be used in combination with a depth filtration, AEX or HIC step after the Protein A capture step to polish the antibody process stream. The viral inactivation step, if not performed prior to the Protein A capture step, can be done after the Protein A but before depth filtration and other chromatographic fine purifications operations.

Certain embodiments of the present invention will include further purification steps. Examples of additional purification procedures which can be performed prior to, during, or following the ion exchange chromatography method include ethanol precipitation, isoelectric focusing, reverse phase HPLC, chromatography on silica, chromatography on heparin Sepharose™, further anion exchange chromatography and/or further cation exchange chromatography, chromatofocusing, SDS-PAGE, ammonium sulfate precipitation, hydroxylapatite chromatography, gel electrophoresis, dialysis, and affinity chromatography (e.g., using protein G, an antibody, a specific substrate, ligand or antigen as the capture reagent).

### 4.5. Methods of Assaying Sample Purity

### 4.5.1 Assaying Host Cell Protein

The present invention also provides methods for determining the residual levels of host cell protein (HCP) concentration in the isolated/purified antibody composition. As described above, HCPs are desirably excluded from the final target substance product. Exemplary HCPs include proteins originating from the source of the antibody production. Failure to identify and sufficiently remove HCPs from the target antibody may lead to reduced efficacy and/or adverse subject reactions.

As used herein, the term “HCP ELISA” refers to an ELISA where the second antibody used in the assay is specific to the HCPs produced from cells, e.g., CHO cells, used to generate the antibody of interest. The second antibody may be produced according to conventional methods known to those of skill in the art. For example, the second antibody may be produced using HCPs obtained by sham production and purification runs, i.e., the same cell line used to produce the antibody of interest is used, but the cell line is not transfected with antibody DNA. In an exemplary embodiment, the second antibody is produced using HCPs similar to those expressed in the cell expression system of choice, i.e., the cell expression system used to produce the target antibody.

Generally, HCP ELISA comprises sandwiching a liquid sample comprising HCPs between two layers of antibodies, i.e., a first antibody and a second antibody. The sample is incubated during which time the HCPs in the sample are captured by the first antibody, for example, but not limited to goat anti-CHO, affinity purified (Cygnus). A labeled second antibody, or blend of antibodies, specific to the HCPs produced from the cells used to generate the antibody, e.g., anti-CHO HCP Biotinylated, is added, and binds to the HCPs within the sample. In certain embodiments the first and second antibodies are polyclonal antibodies. In certain aspects the first and second antibodies are blends of polyclonal antibodies raised against HCPs. The amount of HCP contained in the sample is determined using the appropriate test based on the label of the second antibody.

HCP ELISA may be used for determining the level of HCPs in an antibody composition, such as an eluate or flow-through obtained using the process described above. The present invention also provides a composition comprising an antibody, wherein the composition has no detectable level of HCPs as determined by an HCP Enzyme Linked Immunosorbent Assay (“ELISA”).

### 4.5.2 Assaying Affinity Chromatographic Material

In certain embodiments, the present invention also provides methods for determining the residual levels of affinity chromatographic material (e.g. protein A ligand) in the isolated/purified antibody composition. In certain contexts such material leaches into the antibody composition during the purification process. In certain embodiments, an assay for identifying the concentration of Protein A in the isolated/purified antibody composition is employed. As used herein, the term “Protein A ELISA” refers to an ELISA where the second antibody used in the assay is specific to the Protein A employed to purify the antibody of interest. The second antibody may be produced according to conventional methods known to those of skill in the art. For example, the second antibody may be produced using naturally occurring or recombinant Protein A in the context of conventional methods for antibody generation and production.

Generally, Protein A ELISA comprises sandwiching a liquid sample comprising Protein A (or possibly containing Protein A) between two layers of anti-Protein A antibodies, i.e., a first anti-Protein A antibody and a second anti-Protein A antibody. The sample is exposed to a first layer of anti-Protein A antibody, for example, but not limited to polyclonal antibodies or blends of polyclonal antibodies, and incubated for a time sufficient for Protein A in the sample to be captured by the first antibody. A labeled second antibody, for example, but not limited to polyclonal antibodies or blends of polyclonal antibodies, specific to the Protein A is then added, and binds to the captured Protein A within the sample. Additional non-limiting examples of anti-Protein A antibodies useful in the context of the instant invention include chicken anti-Protein A and biotinylated anti-Protein A antibodies. The amount of Protein A contained in the sample is determined using the appropriate test based on the label of the second antibody. Similar assays can be employed to identify the concentration of alternative affinity chromatographic materials.

Protein A ELISA may be used for determining the level of Protein A in an antibody composition, such as an eluate or flow-through obtained using the process described in above. The present invention also provides a composition comprising an antibody, wherein the composition has no detectable level of Protein A as determined by a Protein A Enzyme Linked Immunosorbent Assay (“ELISA”).

### 4.6. Further Modifications

The antibodies of the present invention can be modified. In some embodiments, the antibodies are chemically modified to provide a desired effect. For example, pegylation of antibodies or antibody fragments of the invention may be carried out by any of the pegylation reactions known in the art, as described, e.g., in the following references: Focus on Growth Factors 3:4-10 (1992); EP 0 154 316; and EP 0 401 384, each of which is incorporated by reference herein in its entirety. In one aspect, the pegylation is carried out via an acylation reaction or an alkylation reaction with a reactive polyethylene glycol molecule (or an analogous reactive water-soluble polymer). A suitable water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (Cl—ClO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under suitable conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

Generally the pegylated antibodies and antibody fragments have increased half-life, as compared to the nonpegylated antibodies and antibody fragments. The pegylated antibodies and antibody fragments may be employed alone, together, or in combination with other pharmaceutical compositions.

An antibody of the invention can be derivatized or linked to another functional molecule (e.g., another peptide or protein). For example, an antibody of the invention can be functionally linked (by chemical coupling, genetic fusion, noncovalent association or otherwise) to one or more other molecular entities, such as another antibody (e.g., a bispecific antibody or a diabody), a detectable agent, a cytotoxic agent, a pharmaceutical agent, and/or a protein or peptide that can mediate associate of the antibody with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

Useful detectable agents with which an antibody of the invention may be derivatized include fluorescent compounds. Exemplary fluorescent detectable agents include fluorescein, fluorescein isothiocyanate, rhodamine, 5-dimethylamine-1-napthalenesulfonyl chloride, phycoerythrin and the like. An antibody may also be derivatized with detectable enzymes, such as alkaline phosphatase, horseradish peroxidase, glucose oxidase and the like. When an antibody is derivatized with a detectable enzyme, it is detected by adding additional reagents that the enzyme uses to produce a detectable reaction product. For example, when the detectable agent horseradish peroxidase is present, the addition of hydrogen peroxide and diaminobenzidine leads to a colored reaction product, which is detectable. An antibody may also be derivatized with biotin, and detected through indirect measurement of avidin or streptavidin binding.

### 4.7. Pharmaceutical Compositions

The antibodies and antibody-binding portions thereof, of the invention can be incorporated into pharmaceutical compositions suitable for administration to a subject. Typically, the pharmaceutical composition comprises an antibody of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it is desirable to include isotonic agents, e.g., sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody.

The antibodies and antibody-binding portions thereof, of the invention can be incorporated into a pharmaceutical composition suitable for parenteral administration. The antibody or antibody-portions can be prepared as an injectable solution containing, e.g., 0.1-250 mg/mL antibody. The injectable solution can be composed of either a liquid or lyophilized dosage form in a flint or amber vial, ampule or pre-filled syringe. The buffer can be L-histidine approximately 1-50 mM, (optimally 5-10 mM), at pH 5.0 to 7.0 (optimally pH 6.0). Other suitable buffers include but are not limited to sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 24%). Stabilizers can be used in both liquid and lyophilized dosage forms, principally 1-50 mM L-methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants.

In one aspect, the pharmaceutical composition includes the antibody at a dosage of about 0.01 mg/kg-10 mg/kg. In another aspect, the dosages of the antibody include approximately 1 mg/kg administered every other week, or approximately 0.3 mg/kg administered weekly. A skilled practitioner can ascertain the proper dosage and regime for administering to a subject.

The compositions of this invention may be in a variety of forms. These include, e.g., liquid, semi-solid and solid dosage forms, such as liquid solutions (e.g., injectable and infusible solutions), dispersions or suspensions, tablets, pills, powders, liposomes and suppositories. The form depends on, e.g., the intended mode of administration and therapeutic application. Typical compositions are in the form of injectable or infusible solutions, such as compositions similar to those used for passive immunization of humans with other antibodies. One mode of administration is parenteral (e.g., intravenous, subcutaneous, intraperitoneal, intramuscular). In one aspect, the antibody is administered by intravenous infusion or injection. In another aspect, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the methods of preparation are vacuum drying and spray-drying that yields a powder of the active ingredient plus any additional desired ingredient from a previously sterile-filtered solution thereof. The proper fluidity of a solution can be maintained, e.g., by the use of a coating such as lecithin, by the maintenance of the required particle size in the case of dispersion and by the use of surfactants. Prolonged absorption of injectable compositions can be brought about by including in the composition an agent that delays absorption, e.g., monostearate salts and gelatin.

The antibodies and antibody-binding portions thereof, of the present invention can be administered by a variety of methods known in the art, one route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978, the entire teaching of which is incorporated herein by reference.

In certain aspects, an antibody or antibody-binding portion thereof, of the invention may be orally administered, e.g., with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain aspects, an antibody or antibody-binding portion thereof, of the invention is co-formulated with and/or co-administered with one or more additional therapeutic agents that are useful for treating disorders. Such combination therapies may advantageously utilize lower dosages of the administered therapeutic agents, thus avoiding possible toxicities or complications associated with the various monotherapies. It will be appreciated by the skilled practitioner that when the antibodies of the invention are used as part of a combination therapy, a lower dosage of antibody may be desirable than when the antibody alone is administered to a subject (e.g., a synergistic therapeutic effect may be achieved through the use of combination therapy which, in turn, permits use of a lower dose of the antibody to achieve the desired therapeutic effect).

It should be understood that the antibodies of the invention can be used alone or in combination with an additional agent, e.g., a therapeutic agent, said additional agent being selected by the skilled artisan for its intended purpose. For example, the additional agent can be a therapeutic agent art-recognized as being useful to treat the disease or condition being treated by the antibody of the present invention. The additional agent also can be an agent which imparts a beneficial attribute to the therapeutic composition, e.g., an agent which affects the viscosity of the composition.

### 5. EXAMPLES

### 5.1. Examples 1

### Effect of MAb Concentration and Kosmotropic Salts on Static Binding Capacity of MabSelect SuRe Protein A Resin for Canine MAb A

The static binding capacity (Qs) of MabSelect SuRe Protein A resin for a Canine MAb A was measured at various feed concentration and salt conditions. In one experiment, a semi-purified canine MAb feed was used to evaluate the Qs values for the resin at different protein concentration. 500 ul of 20% MabSelect SuRe resin slurry was first transferred into a 7 mL size filter column. The resin was washed with 2 mL of water, followed by 2 mL of 0.1 M acetic acid pH 3.5 solution, 4 mL of water and then 5 mL of equilibration buffer which consisted of 50 mM Tris, 100 mM NaCl at pH 7.0. The canine MAb A feed was conditioned to ˜pH 7.1 and conductivity ˜11.6 mS/cm with final concentration ranging from 0.9 to 4.5 g/L. The resin was incubated with 1.9 to 4.5 mL of each feed on a rotating mixed for 2 hours at room temperature. After adsorption, the resin-protein slurries were filtered and the filtrates were collected. The resins were then washed with 2 mL of equilibration buffer followed by incubation with 2 mL of 20 mM Tris, pH 8.5, 0.6 mS/cm elution buffer for 30 min. The resin slurries were filtered again and filtrate collected into clean tube. The resin was then rinsed with 1 mL of elution buffer and the filtrate was collected and combined with the first eluate sample. These eluate samples were then measured by UV280 and Poros G HPLC assays to determine the canine MAb concentration. The Qs values were calculated based on the measured concentrations.

In another set of experiment, 500 ul of 20% MabSelect SuRe resin slurry was first transferred into a 7 mL size filter column. The resin was washed with 2 mL of water, followed by 2 mL of 0.1 M acetic acid pH 3.5 buffer, 4 mL of water and then 5 mL of various equilibration buffer. The equilibration buffer consisted of 40 mM Tris at pH 7.5 and 0.3 to 1.1 M (NH₄)SO₄, or 0.3 to 0.6 M Na₂SO₄, or 0.3 to 0.6 M NaCitrate, or none of these salts. The resin was equilibrated with each equilibration buffer before contact with a clarified canine MAb A harvest, which was supplemented with the various salts at concentrations identical to those of the equilibration buffer. The protein concentrations in the conditioned feed samples were between 3.2 to 4.7 g/L. The resin was incubated with 2.25 mL of each feed on a rotating mixed for 2 hours at room temperature. After adsorption, the resin-protein slurries were filtered and the filtrates were collected. The resins were then washed with 2 mL of equilibration buffer followed by incubation with 2 mL of 20 mM Tris, pH 8.5, 0.6 mS/cm elution buffer for 30 min. The resin slurries were filtered again and filtrate collected into clean tube. The resin was then rinsed with 1 mL of elution buffer and the filtrate was collected and combined with the first eluate sample. These eluate samples were then measured by Poros G HPLC assays to determine the canine MAb concentration. The Qs values were calculated based on the measured concentrations.

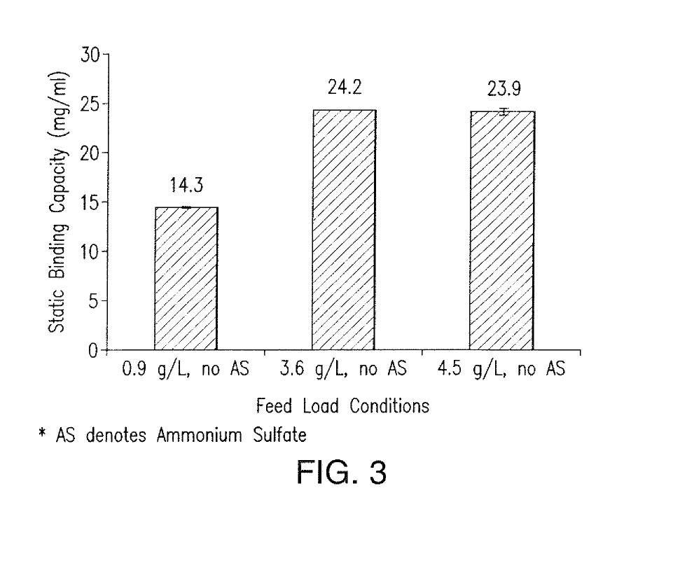

Unlike typical human antibodies, the canine MAb A has significantly lower binding capacity for Protein A, thus its static binding capacity on a standard commercial Protein A resin such as MabSelect SuRe is substantially lower. As shown in FIG. 3, the concentration of this MAb A in the load can significantly affect its Qs on the MabSelect SuRe resin. Increasing MAb A concentration from 0.9 g/L to 4.5 g/L increased the Qs from about 14 g/L to about 24 g/L, although changing the load concentration of 3.6 to 4.5 g/L did not affect Qs value. Thus, pre-concentrating a low titer (e.g. <1 g/L) clarified harvest of canine MAb A should enhance the Protein A binding capacity and throughput during its capture process.

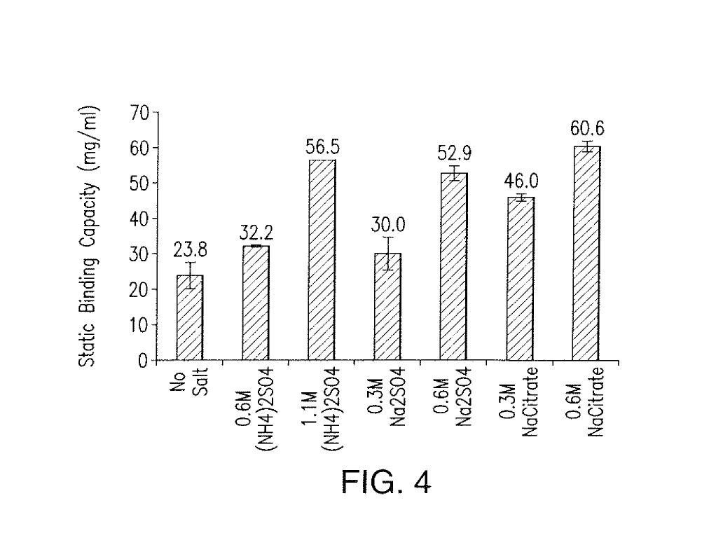

FIG. 4 shows the effects of various kosmotropic salts and their concentrations on the Qs of MabSelect SuRe Protein A resin for canine MAb A. Clearly, adding the kosmotropic salt such as (NH₄)₂SO₄, Na₂SO₄, or NaCitrate increases the Qs values dramatically; and the higher the salt concentration the higher the Qs. In the absence of the salt, the MabSelect SuRe resin gives ˜24 g/L total binding capacity at a feed MAb concentration of 4.7 g/L. In the presence of 1.1 M (NH₄)₂SO₄, the Qs increases to ˜57 g/L at a feed MAb concentration of 4.0 g/L. The latter Qs value reflects a typically observed static binding capacity for a standard, high affinity antibody on the MabSelect SuRe resin (i.e. 50-60 g/L). Consistent with “Hofmeister” series, NaCitrate is the most effective among the three salts in terms of boosting up the Qs at a given salt concentration. The Na₂SO₄ is also more effective than (NH₄)₂SO₄, and it increases Qs to ˜53 g/L at concentration of 0.6 M versus ˜32 g/L for the same concentration of (NH₄)SO₄. Nevertheless, all these salts can be used to effectively enhance the canine MAb A static binding capacity on a Protein A resin.

### 5.2. Example 2

### Effect of MAb Concentration and Ammonium Sulfate on Dynamic Binding Capacity of Canine MAb A on MabSelect SuRe Protein A Resin

The dynamic binding capacity (DBC) of canine MAb A on a MabSelect SuRe Protein A column was first measured using a clarified harvest in the absence of (NH₄)₂SO₄ or other kosmotropic salt. A canine MAb A clarified harvest (initially at ˜1.0 g/L titer) was first concentrated by 8-fold using a 30 kD Biomax membrane cassette. The concentrated harvest was 0.22 um filtered and then diluted with phosphate-buffered saline (PBS) solution to obtain final protein concentration of 0.8-5.6 g/L. These conditioned harvest feeds were used as the load material for MabSelect SuRe column. The column was first equilibrated with PBS buffer followed by feed loading at a flow rate corresponding to 4 min residence time (RT). The flow-through fractions were collected and measured using a Poros G assay to quantify MAb A concentrations which were used to determine the breakthrough curves. After feed loading, the MabSelect SuRe column was washed with equilibration buffer and then eluted with 20 mM Tris, pH 8.5 buffer (This MAb is not stable at low pH so standard low pH elution cannot be used here). The column was then regenerated with 0.15 M phosphoric acid followed by 0.1 M NaOH cleaning before next use.

The DBCs for canine MAb A was also measured in the presence of 1 M (NH₄)₂SO₄. Again, the original canine MAb A clarified harvest (at ˜1.0 g/L titer) was first concentrated by 8-fold using a 30 kD Biomax membrane cassette. The concentrated harvest was diluted with 40 mM Tris, 2.2 M (NH₄)₂SO₄, pH 7.5 solution to obtain final protein concentration of 5.3 g/L and (NH₄)₂SO₄ concentration of 1 M. This material was then 0.22 um filtered to remove haziness. There was no product loss during these preparation steps. The concentrated harvest feed was used to determine the DBC of the MabSelect SuRe resin with 1 M (NH₄)₂SO₄ in the feed and 1.1 M (NH₄)₂SO₄ in the EQ/wash buffer. The DBC run was carried out on MabSelect SuRe column at 4 min and 6 min RT flow rates. In another run, the concentrated feed was also diluted to ˜3 g/L and then diluted with 2.2 M (NH₄)₂SO₄ to obtain 1 M (NH₄)₂SO₄ and final MAb concentration of 1.7 g/L, and the DBC of MabSelect SuRe resin at 6 min RT was determined with this material. The flow-through fractions during each run were collected and analyzed by Poros G assay to determine the breakthrough curve. The column elution and regeneration were identical to those described above.

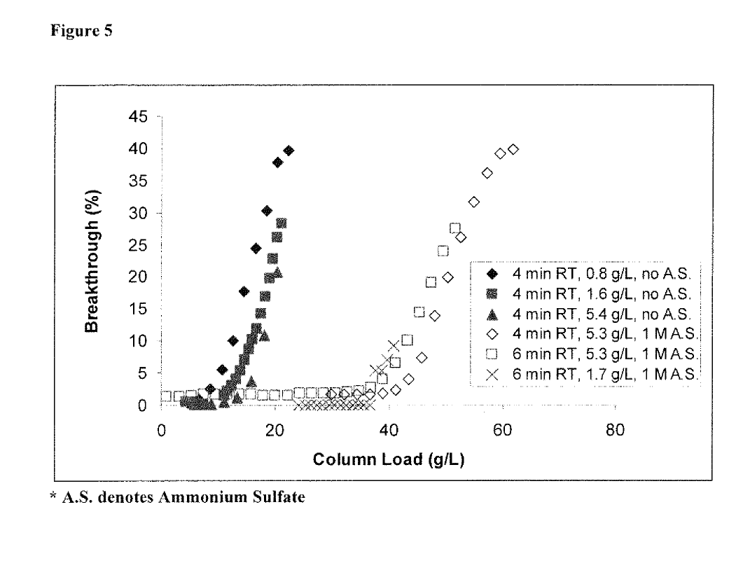

FIG. 5 shows the breakthrough curves for canine MAb A on MabSelect SuRe Protein A column in the absence and presence of (NH₄)₂SO₄ and at various MAb concentration and RT. When there was no (NH₄)₂SO₄ in the load sample, the protein breakthrough occurred much earlier (i.e. <20 g/L resin load), and increasing MAb concentration in the load delayed the breakthrough, consistent with Qs data shown in Example 1. In comparison, adding 1 M (NH₄)₂SO₄ in the load is much more effective in increasing DBCs as the breakthrough curves shifted to much higher column loading level. The breakthrough curves were not significantly affected by the MAb concentration in the range of 1.7 to 5.3 g/L or the flow residence time from 4 to 6 min. The measured DBC values were summarized in Table 2. Overall, the DBC of canine MAb A on MabSelect SuRe column increased about 4 fold by increasing protein concentration from 0.8 g/L to 5.4 g/L and by adding 1 M (NH₄)₂SO₄ into the harvest load.

TABLE 2
Effect of MAb Concentration, Flow Rate and (NH₄)₂SO₄ on
Dynamic Binding Capacities of Canine MAb A on MabSelect SuRe Resin.

| Load Conditions | | | |
|---|---|---|---|
| MAb A Conc. (g/L) | (NH₄)₂SO₄ (M) | RT (min) | DBC (5% BT, g/L) |
| 0.8 | 0 | 4 | 10 |
| 1.6 | 0 | 4 | 13.6 |
| 5.4 | 0 | 4 | 16 |
| 5.3 | 1 | 4 | 44 |
| 5.3 | 1 | 6 | 41 |
| 1.7 | 1 | 6 | 38 |

### 5.3. Example 3

### Effect of Various Kosmotropic Salt on Dynamic Binding Capacity of Canine MAb A on MabSelect SuRe Protein A Resin

Apart from (NH₄)₂SO₄, Na₂SO₄ and NaCitrate were also evaluated in DBC experiments for canine MAb A on the MabSelect SuRe resin. The feed preparation was similar to that described in Example 2, except that the concentrated clarified harvest was supplemented with a concentrated Na₂SO₄ or NaCitrate stock solution to obtain final salt concentration of 0.5 or 0.3 M and protein concentration of 4.8-5.5 g/L. For comparison, a condition at 0.5 M (NH₄)₂SO₄ at similar protein concentration was also conducted in this set of runs. The DBC experiments were performed at flow rate corresponding to 4 to 6 min RT.

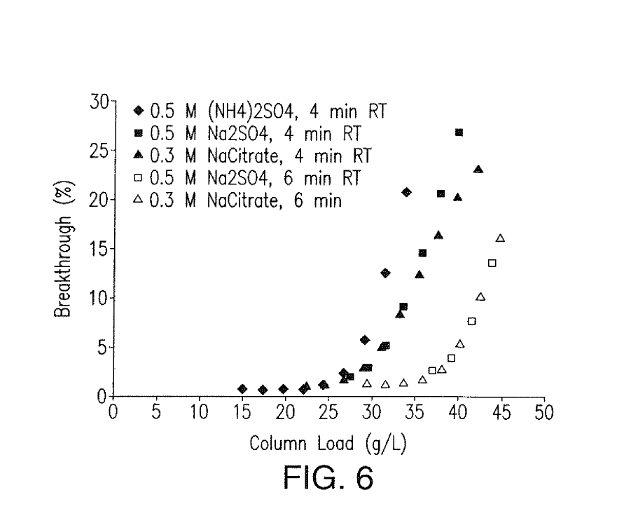

FIG. 6 shows the breakthrough curves for canine MAb A on MabSelect SuRe Protein A resin when the feed contains 0.5 M (NH₄)₂SO₄, 0.5 M Na₂SO₄, or 0.3 M NaCitrate. Consistent with the static binding capacity results, both Na₂SO₄ and NaCitrate give higher DBC than (NH₄)₂SO₄ at the same flow rate and similar salt concentrations. The DBC at 5% breakthrough was 29.1 g/L for 0.5 M (NH₄)₂SO₄, 31.6 g/L for 0.5 M Na₂SO₄ and 31.1 g/L for 0.3 M NaCitrate at 4 min RT flow rate, and 39.2 g/L for 0.5 M Na₂SO₄ and 40.3 g/L for 0.3 M NaCitrate at 6 min RT. Again, it shows that NaCitrate is most effective in enhancing MAb A binding capacity because the higher binding capacity was obtained with the least salt concentration (e.g. 0.3 M). In comparison, a 0.5 M Na₂SO₄ or higher concentration (>0.5 M) of (NH₄)₂SO₄ is needed to achieve similar DBC.

### 5.4. Example 4

### Effect of (NH₄)₂SO₄ Concentration on MabSelect SuRe Protein A Resin Performance for Canine MAb A

The capture performance of MabSelect SuRe Protein A resin was evaluated at various concentrations of (NH₄)₂SO₄ for canine MAb A. The DBC experiments were assessed at (NH₄)₂SO₄ concentration of 0 to 1 M. In this set of experiments, the equilibration and wash buffer contained the same concentration of (NH₄)₂SO₄ as that in the load sample, which was prepared by pre-concentration of a low titer harvest and supplemented with a stock (NH₄)₂SO₄ solution to get to the targeted salt and protein concentrations (as described in Example 2). The protein concentrations ranged from 4.7 to 5.8 g/L. After equilibration with the respective buffer, the column was loaded with the conditioned feed until breakthrough occurred or slightly before breakthrough. The column was then washed with 6 CV of the equilibration buffer, and then eluted with 5 CV of 20 mM Tris, pH 8.5 solution. The eluate pool was collected based on UV280 from 200 mAU to 200 mAU. The column was then regenerated with 0.15 M phosphoric acid followed by 0.1 N NaOH cleaning before next use. All steps were operated at 4 min RT flow rate. In this case, the eluate pool was collected and analyzed by Poros G assay to determine the protein concentration and by an in-house HCP ELISA assay to quantify the HCP levels. In the case that breakthrough was not occurred, the DBC value should be greater than that determined from the eluate protein concentration.

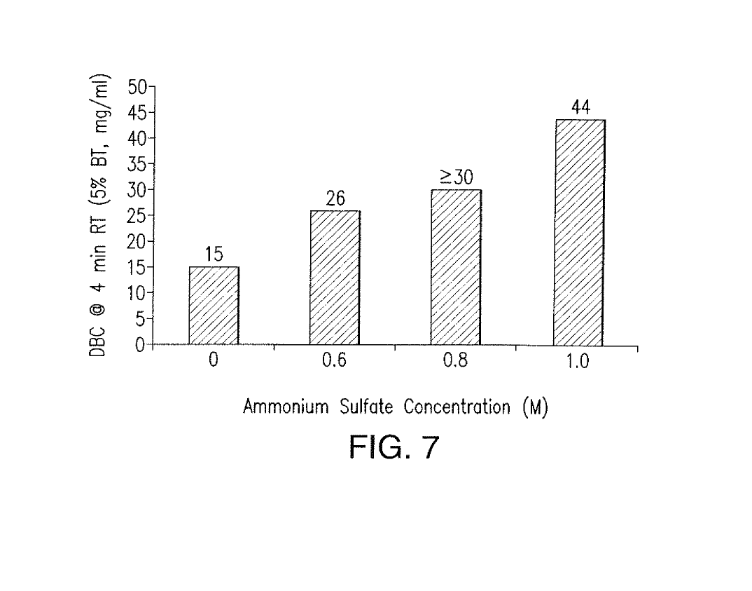

The effect of (NH₄)₂SO₄ concentration on the DBCs of MabSelect SuRe resin was shown in FIG. 7. The differences in the load MAb concentration should have no effect on the DBC, according to results shown in Example 3, thus, the capacity differences observed here were due to the effect of (NH₄)₂SO₄. As expected, increasing (NH₄)₂SO₄ concentration has a large impact on the DBCs for canine MAb A. An approximately 3-fold improvement on the DBC was observed when (NH₄)₂SO₄ concentration increased from 0 to 1 M. Thus, adjusting kosmotropic salt concentration can be used to modulate the binding capacity of a Protein A resin for this weakly associated antibody molecule.

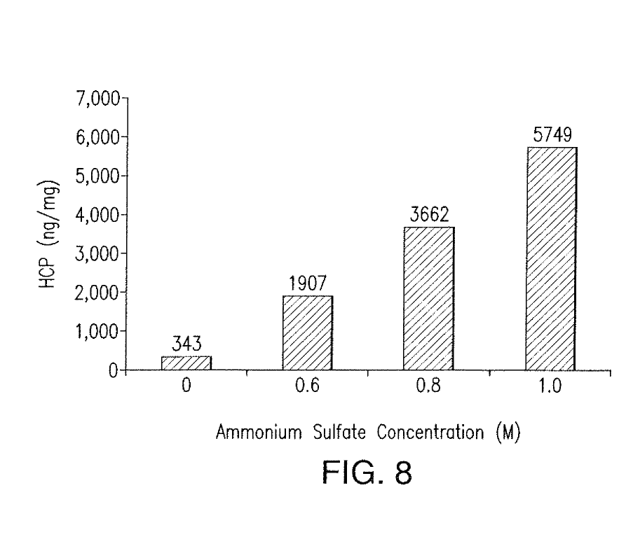

FIG. 8 showed the HCP levels in the eluate pool during MAbSelect SuRe capture purification of the canine MAb A in the presence of various concentrations of (NH₄)₂SO₄. Similar to MAb A, an increased binding of HCP to the resin was also observed as (NH₄)₂SO₄ concentration increased. However, such HCP levels were still within the range typically observed for a MAb on Protein A resin. Selecting an appropriate (NH₄)₂SO₄ concentration is critical to meet both throughput and product quality requirements. Same conclusion can be drawn for other kosmotropic salts given their similar behavior on the binding capacity.

### 5.5. Example 5

### Canine MAb A Purification by a Two-Column Process Based on (NH₄)₄SO₄-Assisted Protein A Capture

A 50 L canine MAb A bioreactor harvest was clarified by using 0.55 m² of DOHC followed by 0.33 m² of X0HC Pod depth filter and 0.1 m² Sartopore 2 0.45/0.2 um sterile filter cartridge. The clarified harvest (˜1.0 g/L titer) was first concentrated by approximately 11-fold using a 30 kD Biomax membrane cassette. The concentrated harvest was diluted to 3 mg/ml, then supplemented with 0.1% (v/v) Triton X-100. It was then diluted with 40 mM Tris, 2.2 M (NH₄)₂SO₄, pH 7.5 solution to obtain final protein concentration of 2.5 g/L and (NH₄)SO₄ concentration of 0.5 M. This material was then 0.22 um filtered to remove haziness.

A 1.0 cm (i.d.)×22 cm MabSelect SuRe column was pre-conditioned with 0.1 N NaOH followed by equilibration with 5 CV of 20 mM Tris, 0.5 M (NH₄)₂SO₄, pH 7.5 buffer. The column was then loaded with the (NH₄)₂SO₄-conditioned harvest (titer 2.5 g/L) to a total loading level of 26 g/L using staged flow rate: 0-20 g/L at 330 cm/hr and 20-26 g/L at 220 cm/hr. The column was then washed with 5 CV of 20 mM Tris, 0.8 M (NH₄)₂SO₄, pH 7.5 buffer followed by 1 CV of 20 mM Tris, 0.5 M (NH₄)₂SO₄, pH 7.5 buffer at 330 cm/hr prior to elution with 5 CV of 20 mM Tris, pH 8.5 buffer. The elution pool was collected based on UV280 from 500 to 500 mAU. After elution, the column was regenerated with 3 CV of 0.15 M phosphoric acid and cleaned with 5 CV of 0.1 M NaOH at 380 cm/hr. The column was re-equilibrated before the next cycle. Five cycles were run to generate enough materials for downstream processing.

The protein A eluates were combined and conditioned to final conductivity of 28 mS/cm and pH 8. The conditioned feed, with total mass of 1.6 g, was then filtered through a 26 cm² X0HC μPod device at ˜100 LMH flow rate. After feed load, the filter was flushed with 52 ml of 20 mM Tris, 0.1 M (NH₄)₂SO₄, pH 8 buffer to recover any bound product.

The filtrate was diluted with 20 mM Tris, pH 8 buffer to achieve conductivity of 6 mS/cm at pH 8 for further polishing through a 5 ml prepacked Capto Q column (GE Healthcare). The column was cleaned with 0.1 N NaOH, equilibrated with 5 CV of 25 mM Tris, 27 mM NaCl, pH 8 (6 mS/cm) buffer, then loaded with the diluted X0HC filtrate to about 40 g/L loading level at staged flow rate (0-33 g/L at 1.25 ml/min and 33-40 g/L at 0.5 ml/min). The column was washed with 8 CV of equilibration buffer and eluted with 50 mM Tris, 280 mM NaCl, pH 7.5 buffer (32.5 mS/cm) at 1.25 ml/min. The elution pool was collected based on UV280 from 200 to 200 mAU. The column was then stripped with 5 CV of 50 mM Tris, 1 M NaCl, pH 7.5 buffer followed by cleaning with 5 CV of 0.5 N NaOH at 2.5 ml/min flow rate.

The eluate or filtrate samples were taken from each step for yield and purity analyses. The protein concentration was measured by UV280 and Poros G assay. The monomer/aggregates levels were determined by SEC, HCP and leached protein A by in-house ELISA assays.

Table 3 summarizes the step yield and impurity level from each step. The step yield for harvest clarification was ˜74%, slightly lower than one would expect. This is due to lack of buffer flush of the filter after loading the harvest sample. The yields for all the other steps were within typical range for the respective operations, and were all above 90%. The MabSelect SuRe column effectively removed the majority of the HCPs, from the initial 200,000 ng/mg in the load to <400 ng/mg in the Protein A eluate, representing a 2.6 log clearance. The X0HC provided additional one log reduction on the HCP level and the Capto Q resin further reduced it to less than 10 ng/mg. The final product has a monomer level over 99% (with aggregates less than 1%) and leached protein A below quantitation limit.

TABLE 3
Purification Performances of a Two-Column Process for
Canine MAb A.

| | Yield | HCP | Monomer | Aggregate | Protein A |
|---|---|---|---|---|---|
| Step | (%) | (ng/mg) | (%) | (%) | (ng/mg) |
| Clarification | 74 | ND | NA | NA | NA |
| MabSelect SuRe | 100 | 158774- | NA | NA | NA |
| Protein A load | | 211622 | | | |
| preparation | | | | | |
| (NH₄)₂SO₄-assisted | 90 | 238-391 | 98.7 | 1.01 | 4.59 |
| MabSelect SuRe | | | | | |
| Protein A capture | | | | | |
| X0HC filtration | 90 | 18 | 98.8 | 0.80 | LTQ * |
| Capto Q bind-elute | 90-95 | 6 | 99.1 | 0.86 | LTQ * |
| polishing | | | | | |

* LTQ denotes less than quantitation limit.

### 5.6. Example 6

### Canine MAb A Purification by a Three-Column Process Based on (NH₄)₂SO₄-Assisted Protein A Capture

The MabSelect SuRe protein A eluate obtained from the experiments shown in Example 5 was also purified through a 5 mL prepacked Capto Phenyl column which was run in flow-through mode. Specifically, the Protein A eluate was first diluted with a 20 mM Tris, pH 7.5 buffer to achieve final conductivity ˜23 mS/cm and MAb concentration ˜10 mg/ml. The Capto Phenyl column was cleaned with 0.1 M NaOH followed by equilibration with 5 CV of 20 mM Tris, 0.1 M (NH₄)₂SO₄, pH 7.5 buffer. The column was then loaded with the diluted feed to 80 g/L loading level at 4 min RT flow rate. After that, the column was washed with 10 CV equilibration buffer at the same flow rate. The flow-through pool was collected during the load when UV280 reached 200 mAU and stopped during the wash when UV280 reading dropped back to 200 mAU.

The Phenyl eluate was then conditioned to pH 8, 6 mS/cm and purified through the Capto Q column as described in Example 5. Again, the eluate samples were taken from each step for yield and purity (HCP and aggregates/monomer) analyses.

Table 4 summarizes the purification performance for this three-column process. In this case, Capto Phenyl column plays the same role in terms of impurity clearance as the X0HC filter shown in Example 5. This resin also provided one log reduction for HCP at high step yield (97%). The final product after the Capto Q polishing step has ˜3 ng/mg HCP and 0.45% aggregates (monomer level 99.5%).

TABLE 4
Purification Performances of a Three-Column Process for Canine MAb A.

| | Yield | HCP | Monomer | Aggregate |
|---|---|---|---|---|
| Step | (%) | (ng/mg) | (%) | (%) |
| Clarification | 74* | ND | NA | NA |
| MabSelect SuRe Protein A | 100 | 158774- | NA | NA |
| load preparation | | 211622 | | |
| (NH₄)₂SO₄-assisted | 104 | 552 | 99.0 | 0.87 |
| MabSelect SuRe Protein | | | | |
| A capture | | | | |
| Capto Phenyl flow-through | 97 | 51 | 99.2 | 0.64 |
| Capto Q bind-elute polishing | 93 | 3 | 99.5 | 0.45 |

### 5.7. Example 7. Canine MAb A Purification by an Alternative Two-Column Process Based on Na₂SO₄-Assisted Protein A Capture

A two-column process alternative to that described in Example 5 was used to purify canine MAb A. The major difference for this process was the use of Na₂SO₄ instead of (NH₄)₂SO₄ in the MabSelect SuRe Protein A operation. The pre-concentrated canine MAb A (as described in Example 5) was supplemented with 0.05% Triton X-100 and then 0.5 M Na₂SO₄; the protein concentration was adjusted to 5.8 g/L. The 1.0 cm (i.d.)×22 cm MabSelect SuRe column was pre-conditioned with 0.1 N NaOH followed by equilibration with 5 CV of 20 mM Tris, 0.8 M Na₂SO₄, pH 7.5 buffer. The column was then loaded with the Na₂SO₄-conditioned harvest to a total loading level of −44 g/L using staged flow rate: 0-24 g/L at 335 cm/hr and 24-44 g/L at 220 cm/hr. The column was then washed with up to 6 CV of 20 mM Tris, 0.8 M Na₂SO₄, pH 7.5 buffer prior to elution with 5 CV of 20 mM Tris, pH 8.5 buffer. The elution pool was collected based on UV280 from 500 to 500 mAU. The column regeneration and cleaning steps were performed identical to that shown in Example 5.

The Protein A eluates were pooled and adjusted to pH 8 and 29 mS/cm for X0HC filtration step. The actual loading level on the X0HC filter was ˜409 g/m². The X0HC filtrate was then purified through Capto Q column. The operating procedures for both X0HC and Q steps were similar to those shown in Example 5. The samples from each step were analyzed to determine the yield, HCP and monomer/aggregates levels.

Table 5 summarized the performance data for Na₂SO₄-based two-column process. Again, all step recoveries were within expected range. The Na₂SO₄-assisted Protein A step allows high loading level but resulted in higher HCP, as one would have expected. This relatively higher HCP level in the MabSelect SuRe eluate can be effectively reduced by the X0HC and Capto Q polishing steps. The final product contained ˜28 ng/mg HCP and ˜1.5% aggregates. The increased aggregate levels in X0HC filtrate and Capto Q elute were due to sample aging for extended period of time before proper SEC analysis was run. Nevertheless, the product quality is within acceptable range for this molecule.

TABLE 5
Purification Performances of an Alternative Two-Column Process
for Canine MAb A.

| | Yield | HCP | Monomer | Aggregate |
|---|---|---|---|---|
| Step | (%) | (ng/mg) | (%) | (%) |
| Clarification | 74* | ND | NA | NA |
| MabSelect SuRe Protein | 100 | 158774- | NA | NA |
| A load preparation | | 211622 | | |
| Na₂SO₄-assisted MabSelect | 93-105 | 1862-2531 | 96.5-96.9 | 1.0-1.1 |
| SuRe Protein A capture | | | | |
| X0HC filtration | 94 | 616 | 97.8* | 1.6* |
| Canto Q bind-elute polishing | 84 | 28 | 98.2* | 1.5* |

*material aged prior to SEC analysis

### 5.8. Example 8

### Dynamic Binding Capacity of Canine MAb A on ProSep Ultra Plus Protein A Resin

The DBC of canine MAb A on a ProSep Ultra Plus Protein A (PUP) column was measured using a purified canine MAb A feed in the absence of kosmotropic salt, or in the presence of 1M (NH₄)₂SO₄, 0.3M sodium citrate (NaCitrate) or 0.5M Na₂SO₄. In these experiments, the canine MAb A feed concentration was adjusted to 2.6-2.8 g/L. A 1 mL pre-packed PUP protein A column was first equilibrated with 20 mM Tris, pH 7.5 buffer (for the case of no salt addition) or 20 mM Tris, pH 7.5 buffer supplemented with 1M (NH₄)₂SO₄, or 0.3M sodium citrate, or 0.5M Na₂SO₄, respectively, followed by feed loading at a flow rate corresponding to 3 min residence time (RT). The breakthrough curves were monitored at UV280 and the DBC values at 5% BT were determined accordingly. After feed loading, the PUP column was washed with respective equilibration buffer and then eluted with a 20 mM Tris, pH 8.5 buffer. The column was then regenerated with 0.15 M phosphoric acid before next use.

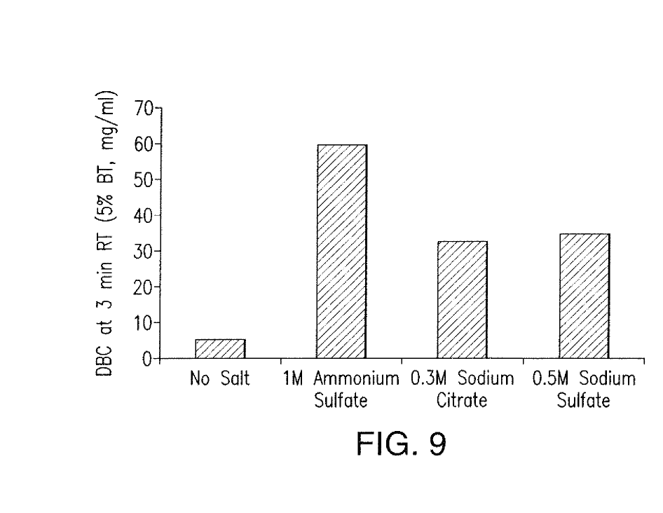

FIG. 9 compares the DBC values for canine MAb A on PUP Protein A column in the absence and presence of various kosmotropic salts at 3 min RT. When there was no salt in the load sample, the canine MAb A capacity was only about 5 g/L resin. In contrast, the DBC increased by over 10-fold when adding 1M (NH₄)₂SO₄ in the load, or increased by over 6-fold when adding 0.3 M Na₂SO₄ or 0.5 M NaCitrate in the load. This data confirm that the increase of canine MAb binding affinity by using kosmotropic salt is independent of the protein A resin used.

### 5.9. Example 9

### Dynamic Binding Capacity of Human MAb 1 and MAb 2 on ProSep Ultra Plus Protein A Resin

The DBCs of a human MAb 1 and MAb 2 on a ProSep Ultra Plus Protein A (PUP) column were measured using purified MAb 1 or MAb 2 feed in the absence or presence of 1M (NH₄)₂SO₄. Both feed concentrations were adjusted to −1.7 g/L. A 5 mL PUP protein A column was first equilibrated with 20 mM Tris, pH 7.5 buffer (for the case of no salt addition) or 20 mM Tris, 1M (NH₄)₂SO₄, pH 7.5 buffer followed by feed loading at a flow rate corresponding to 3 min RT. The breakthrough curves were monitored at UV280 and the DBC values at 5% BT were determined accordingly. After feed loading, the PUP column was washed with the respective equilibration buffer and then eluted with 0.1M acetic acid, pH 3.5 buffer. The column was then regenerated with 0.15 M phosphoric acid before next use.

Table 6 compares the DBC values for human MAb 1 and MAb 2 on PUP Protein A column in the absence and presence of (NH₄)₂SO₄ at 3 min RT. When there was no salt in the load sample, the DBC was about 58 and 64 g/L resin for MAb 1 and MAb 2, respectively. Adding 1 M (NH₄)₂SO₄ increased it to about 69 g/L for MAb 1 or 71 g/L for MAb 2. This data indicates that using kosmotropic salt can further increase the Protein A resin binding capacity for human antibodies which have much higher affinity for the Protein A ligand.

TABLE 6
Comparison of DBCs for human MAbs on ProSep Ultra Plus
Protein A resin in the absence and presence of (NH₄)₂SO₄

| | DBC at 5% BT (mg/ml) | |
|---|---|---|
| Molecules | No Salt | 1M (NH₄)₂SO₄ |
| Human MAb 1 | 58 | 69 |
| Human MAb 2 | 64 | 71 |

### 5.10. Example 10

### Effect of (NH₄)SO₄ on Purification of Human MAb 1 on ProSep Ultra Plus Protein A Column

The effect of (NH₄)₂SO₄ on capture purification performance of PUP resin for human MAb 1 was further assessed using clarified harvest. In this set of experiments, the titer of MAb 1 clarified harvest was adjusted to 1.1-1.2 g/L, with or without 1M (NH₄)₂SO₄. The 5 mL PUP column was equilibrated with 20 mM Tris, pH 7.5 buffer (for the case of no salt) or 20 mM Tris, pH 7.5, 1M (NH₄)₂SO₄ buffer, then loaded with the respective MAb 1 clarified harvest to 50 g/L (for the case of no salt) and 59 g/L (for LM (NH₄)₂SO₄ case) resin loading level, respectively, washed with the respective EQ buffer followed by 20 mM sodium citrate, 0.5M NaCl, pH 6 buffer and EQ buffer again, and then eluted using 0.1 M acetic acid, pH 3.5 buffer. The elution pool was collected based on UV280 from 200 mAU to 200 mAU. The column was regenerated with 0.15 M phosphoric acid, cleaned with 0.4 M acetic acid, 0.5 M NaCl, 0.1% Tween 80, pH 2.4 buffer before next use. Duplicated runs were performed at each condition. The elution pool was analyzed for protein concentration, HCP level by in-house ELISA assay and aggregate/monomer level by SEC.

Table 7 summarizes the purification yield and quality results from this experiment. The yield and aggregate/monomer profiles are quite comparable for the two conditions, i.e. with or without (NH₄)₂SO₄, although about 18% higher resin loading level was applied for the PUP column for the case of 1M (NH₄)₂SO₄ run. However, there was a substantial difference in HCP level; when the 1 M (NH₄)₂SO₄ was used the product pool HCP level was 4-5 fold lower than that in the absence of (NH₄)₂SO₄. Thus, adding kosmotropic salt in the clarified harvest not only can enhance Protein A binding capacity for this human MAb but also improve the clearance of process-related impurities such as HCP.

TABLE 7
Purification performance for human MAb 1 on PUP Protein A
column in the absence and presence of (NH₄)₂SO₄

| | Yield | HCP | Aggregates | Monomer |
|---|---|---|---|---|
| Conditions | (%) | (ng/mg) | (%) | (%) |
| No Salt | 92; 91 | 850; 935 | 3.7; 1.7 | 95.7; 98.3 |
| 1M (NH₄)₂SO₄ | 94; 95 | 181; 161 | 2.6; 3.0 | 97.4; 96.8 |

### 5.11. Example 11

### Effect of (NH₄)₂SO₄ on Purification of Human MAb 2 on ProSep Ultra Plus Protein A Column

The effect of (NH₄)₂SO₄ on capture purification performance of PUP resin for human MAb 2 was also assessed using clarified harvest. In this set of experiments, the titer of MAb 2 clarified harvest was adjusted to about 1.2 g/L, with or without 1M (NH₄)₂SO₄. The 5 mL PUP column was equilibrated with 20 mM Tris, pH 7.5 buffer (for the case of no salt) or 20 mM Tris, pH 7.5, 1M (NH₄)₂SO₄ buffer, then loaded with the respective MAb 1 clarified harvest to 57 g/L (for no salt case) and 64 g/L (for 1M (NH₄)₂SO₄ case) resin loading level, respectively, washed with the respective EQ buffer followed by 20 mM sodium citrate, 0.5M NaCl, pH 6 buffer and EQ buffer again, and then eluted using 0.1 M acetic acid, pH 3.5 buffer. The elution pool was collected based on UV280 from 200 mAU to 200 mAU. The column was regenerated with 0.15 M phosphoric acid, cleaned with 0.4 M acetic acid, 0.5 M NaCl, 0.1% Tween 80, pH 2.4 buffer before next use. Duplicated runs were conducted at each condition. The elution pool was analyzed for protein concentration, HCP level by in-house ELISA assay and aggregate/monomer level by SEC.

Table 8 summarizes the purification yield and quality results for MAb 2. Again, the yield and aggregate/monomer profiles are quite comparable for these two runs with or without 1M (NH₄)₂SO₄, although about 12% higher resin loading level was applied for the PUP column in the case of 1M (NH₄)₂SO₄ run. Similar to MAb 1, the product pool HCP level in the presence of 1M (NH₄)₂SO₄ was about 2-3 fold lower than that in the absence of salt. Overall, using kosmotropic salt in the clarified harvest can improve the Protein A capture performance for human or humanized MAbs in addition for canine MAbs.

TABLE 8
Purification performance for human MAb 2 on PUP Protein A
column in the absence and presence of (NH₄)₂SO₄

| | Yield | HCP | Aggregates | Monomer |
|---|---|---|---|---|
| Conditions | (%) | (ng/mg) | (%) | (%) |
| No Salt | 92; 90 | 1230; 1362 | 0.5 | 99.1 |
| 1M (NH₄)₂SO₄ | 90; 91 | 505; 354 | 0.2 | 99.4 |

Various publications are cited herein, the contents of which are hereby incorporated by reference in their entireties.

What is claimed is:

1. A method for producing a host cell-protein (HCP)-reduced antibody preparation from a sample mixture comprising an antibody and at least one HCP, said method comprising:
(a) subjecting said sample matrix to a kosmotropic salt solution thus forming a primary recovery sample;
(b) contacting said primary recovery sample to a Protein A affinity chromatography resin and obtaining a Protein A affinity chromatography eluate sample,
wherein said Protein A affinity chromatography sample comprises an HCP-reduced antibody preparation.

2. The method of claim 1, wherein said antibody has weak binding strength and low binding capacity for the affinity chromatography resin.

3. The method of claim 1, wherein said antibody is a human antibody, humanized antibody, a chimeric antibody, a multivalent antibody, a dual-variable domain antibody, or an antigen-binding portion thereof.

4. The method of claim 1, wherein said antibody in said primary recovery sample that is contacted to said affinity chromatography resin is concentrated such that it has a concentration of from about 1 g/L to about 10 g/L.

5. The method of claim 1, wherein the kosmotropic salt in said kosmotropic salt solution is selected from the group consisting of ammonium sulfate, sodium sulfate, sodium citrate, potassium sulfate, potassium phosphate, sodium phosphate, and a combination thereof.

6. The method of claim 5, wherein said kosmotropic salt is present in said kosmotropic salt solution at a concentration of from about 0.3 M to about 1.1 M.

7. The method of claim 1, wherein said Protein A affinity chromatography resin is selected from any commercial Protein A resins including MabSelect SuRe™, MabSelect, MabSelect SuRe LX, MabSelect Xtra, rProtein A Sepharose Fast Flow, Poros® MabCapture A, Amsphere™ Protein A JWT203, ProSep HC, ProSep Ultra, and ProSep Ultra Plus.

8. The method of claim 1, comprising contacting said affinity chromatography eluate sample to:
(a) an ion exchange media and obtaining an ion exchange eluate sample, wherein said ion exchange eluate sample comprises an HCP-reduced antibody preparation;
(b) a hydrophobic interaction chromatography (HIC) media and obtaining a HIC eluate sample, wherein said HIC eluate sample comprises an HCP-reduced antibody preparation; or
(c) a depth filter and obtaining a filtrated sample.

9. A method for producing a host cell-protein (HCP)-reduced antibody preparation from a sample mixture comprising an antibody and at least one HCP, said method comprising:
(a) concentrating the said sample matrix to obtain a conditioned sample matrix;
(b) contacting said conditioned sample matrix to a Protein A affinity chromatography resin and obtaining a Protein A affinity chromatography eluate sample, wherein said antibody in said conditioned sample matrix that is contacted to said Protein A affinity chromatography resin has a concentration of from about 1 g/L to about 10 g/L; and
wherein said Protein A affinity chromatography eluate sample comprises an HCP-reduced antibody preparation.

10. The method of claim 9, wherein said antibody has weak binding strength and low binding capacity for the affinity chromatography resin.

11. The method of claim 9, wherein said antibody is a human antibody, humanized antibody, a chimeric antibody, a multivalent antibody, a dual-variable domain antibody, or an antigen-binding portion thereof.

12. The method of claim 9, wherein said Protein A affinity chromatography resin is selected from any commercial Protein A resins including MabSelect SuRe™, MabSelect, MabSelect SuRe LX, MabSelect Xtra, rProtein A Sepharose Fast Flow, Poros® MabCapture A, Amsphere™ Protein A JWT203, ProSep HC, ProSep Ultra, and ProSep Ultra Plus.

13. The method of claim 9, comprising contacting said affinity chromatography eluate sample to:
(a) an ion exchange media and obtaining an ion exchange eluate sample, wherein said ion exchange eluate sample comprises an HCP-reduced antibody preparation;
(b) a hydrophobic interaction chromatography (HIC) media and obtaining a HIC eluate sample, wherein said HIC eluate sample comprises an HCP-reduced antibody preparation; or
(c) a depth filter and obtaining a filtrated sample.

14. A pharmaceutical composition comprising an HCP-reduced antibody preparation produced by the method of claim 1, and a pharmaceutically acceptable carrier.

15. A method for improving HCP clearance during Protein A capture purification of an antibody from a mixture comprising the antibody of interest and at least one HCP, said method comprising:
(a) subjecting said sample matrix to a kosmotropic salt solution thus forming a primary recovery sample;
(b) contacting said primary recovery sample to a Protein A affinity chromatography resin and obtaining a Protein A affinity chromatography eluate sample,
wherein said Protein A affinity chromatography eluate sample comprises significantly reduced HCP level as compared to that obtained from the typical Protein A capture process in which a kosmotropic salt solution is not used.

16. The method of claim 15, wherein said antibody is a human antibody, humanized antibody, a chimeric antibody, a multivalent antibody, a dual-variable domain antibody, or an antigen-binding portion thereof.

17. The method of claim 15, wherein the kosmotropic salt in said kosmotropic salt solution is selected from the group consisting of ammonium sulfate, sodium sulfate, sodium citrate, potassium sulfate, potassium phosphate, sodium phosphate, and a combination thereof.

18. The method of claim 17, wherein said kosmotropic salt is present in said kosmotropic salt solution at a concentration of from about 0.3 M to about 1.1 M.

19. The method of claim 15, wherein said Protein A affinity chromatography resin is selected from any commercial Protein A resins including MabSelect SuRe™, MabSelect, MabSelect SuRe LX, MabSelect Xtra, rProtein A Sepharose Fast Flow, Poros® MabCapture A, Amsphere™ Protein A JWT203, ProSep HC, ProSep Ultra, and ProSep Ultra Plus.

20. The method of claim 15 comprising contacting said affinity chromatography eluate sample to:
(a) an ion exchange media and obtaining an ion exchange eluate sample, wherein said ion exchange eluate sample comprises an HCP-reduced antibody preparation;
(b) a hydrophobic interaction chromatography (HIC) media and obtaining a HIC eluate sample, wherein said HIC eluate sample comprises an HCP-reduced antibody preparation; or
(c) a depth filter and obtaining a filtrated sample.
